(19) United States
(12) Patent Application Publication
(10) Pub. No.: US 2025/0002979 A1
(43) Pub. Date: Jan. 2, 2025

(54) A Molecular Tool And Use Thereof In A Label-Free Method For Detecting A Target Nucleic Acid In A Sample

(71) Applicant: ECOLE POLYTECHNIQUE FEDERALE DE LAUSANNE (EPFL), Lausanne (CH)

(72) Inventors: Alice Judith GILLEN, Livermore, CA (US); Ardemis Anoush BOGHOSSIAN, Grandvaux (CH)

(21) Appl. No.: 18/568,145

(22) PCT Filed: Jun. 23, 2022
(86) PCT No.: PCT/EP2022/067129

§ 371 Date: Dec. 7, 2023

(30) Foreign Application Priority Data
Jun. 24, 2021 (EP) 21181418.1

(51) Int. Cl. C12Q 1/6837; C12Q 1/6876

(52) U.S. Cl. CPC C12Q 1/6837; C12Q 1/6876; C12Q 2600/178

(57) ABSTRACT

A molecular tool and a label-free method useful for detecting a target nucleic acid in a sample are disclosed, the molecular tool comprising a substrate comprising or consisting of an optical material; a first polymeric molecule, selected from nucleic acids or nucleic acid analogues, coupled with the substrate; and a second nucleic acid analogue coupled with the first polymeric molecule by means of nucleobase complementarity. The molecular tools and methods of the invention are configured for the optical detection of oligonucleotide binding events for diagnostic, point-of-care, drug screening applications using for instance a carbon nanotube optical signal. Circulating nucleic acids that have diagnostic and prognostic value for cancer, metabolic disease, organ rejection, foetal health and infectious disease can be therefore identified and characterized.

### TECHNICAL FIELD

The present invention generally belongs to the field of molecular diagnostics. In particular, the invention relates generally to the detection of nucleotide sequences (nucleic acids) in a sample through the functionalization of a substrate comprising or consisting of an optical material, such as single-walled carbon nanotubes, for the optical detection of nucleic acids, such as microRNA.

### BACKGROUND ART

Over the past few decades, microRNAs (miRNAs) have emerged as promising biomarkers for a range of diseases. miRNAs are short strands of non-coding RNA, typically 15-25 nucleotides, which are involved in the regulation of many cellular processes including differentiation, cell growth and cell death. These molecules play an important regulatory role in gene expression by triggering degradation, affecting translation, or regulating transcription of target messenger RNAs (mRNAs). Deregulated expression of different miRNAs is associated with a range of diseases, including numerous types of cancers, and as such circulating miRNA and exosomal miRNA has immense potential to serve as non-invasive diagnostic biomarkers.

Despite growing interest in the detection of miRNA, clinical implementation has been limited by the trade-offs typically encountered between accuracy/sensitivity and cost/time for the detection methods currently available. For example, RNA-sequencing provides the most sensitive quantification and detection of miRNA, however this technique requires extensive processing and evaluation steps that increase both the time and cost of analysis. As a result, this technique is typically reserved for use in the discovery of novel miRNA rather than clinical detection. Conversely, the most common method of miRNA detection in diagnostics, reverse-transcription polymerase chain reaction (RT-PCR), relies on relatively standard equipment and the processing can be more or less automated leading to much faster processing times. However, an undeniable bias from unreliable background corrections/normalizations, a reliance on error-prone reverse-transcription reactions, and a lack of standardization for pre-processing steps can lead to sub-optimal specificity and problems with reproducibility when using this approach.

To address aspects of these shortcomings, a new form of immunoassay, miRNA enzyme immunoassay (miREIA), was designed for miRNA quantification. In miREIA, miRNA is hybridized with complementary biotinylated DNA oligonucleotides and these duplexes are subsequently detected using a monoclonal antibody. The miREIA workflow is similar to the ELISA methodology used for antigen detection and can be run on standard immunoassay analyzers increasing its applicability and convenience in clinical settings. In addition, this approach circumvents any need for reverse-transcription or amplification/pre-amplification steps thereby increasing reproducibility. However, as this approach requires miRNA purification from blood samples it has limited use in both unprocessed samples and in vivo applications.

Owing to the many advantages of Single Walled Carbon Nanotubes (SWCNTs), recent works have looked to engineer SWCNT-based optical sensors for the detection of short DNA sequences or miRNA that transduce DNA hybridization into spectral changes in the emission spectrum of the SWCNT. Optical modulation of SWCNT fluorescence was first used to detect DNA hybridization; a hypsochromic shift of 2 meV was observed following the addition of complementary DNA, with no significant wavelength shift observed on addition of a non-complementary strand. The wavelength shifting observed on recognition of the complementary sequence was attributed to an increase in the surface coverage of the SWCNT following hybridization.

Later work went on to demonstrate how these DNA-SWCNTs were also capable of detecting single nucleotide polymorphisms (SNPs). More recent work presented a DNA-SWCNT sensor for the optical detection of different miRNA sequences. Here, spectral changes following hybridization were attributed to the displacement of both electrostatic charge and water from the nanotube surface. Following hybridization, the newly formed duplex was hypothesized to desorb from the surface of the nanotube leaving only the nanotube-binding domain in contact, resulting in an increase of the exposed surface area. Subsequent addition of surfactant molecules led to increased water shielding and a markedly enhanced spectral response as the moieties underwent a triggered assembly on the nanotube surface. These sensors were subsequently used for the direct detection of miRNA in urine and serum and also for the first in vivo optical detection of target DNA and miRNA using SWCNT NIR fluorescence.

XNAs have also been extensively studied for miRNA detection, most notably LNA and peptide nucleic acid (PNA) owing to their improved efficiency compared to DNA probes. PNA is of particular interest due to its uncharged backbone which enables stronger DNA hybridization due to the absence of electrostatic repulsion. An additional consequence of the lack of electrostatic repulsion is that PNA can hybridize with DNA virtually independently of the salt concentration. PNA also demonstrates increased specificity compared to DNA or RNA, as any base pair mismatch results in greater destabilization for PNA than natural oligonucleotides, however the destabilisation of the mismatch is strongly impacted by its position along the sequence.

### SUMMARY OF INVENTION

In order to address and overcome at least some of the above-mentioned drawbacks of the prior art solutions, the present inventors developed a molecular tool as well as a label-free method for detecting a nucleic acid in a sample, having improved features and capabilities.

In particular, a first purpose of the present invention is that of providing a new molecular tool for a label-free detection of a nucleic acid in a sample, such as a biological sample, in a user-friendly and robust setting.

Another purpose of the present invention is that of facilitating the detection of a nucleic acid in a sample through a simple method avoiding processing the sample.

Yet another purpose of the present invention is that of providing a flexible platform, readily adaptable to many different experimental or clinical setting, for the rapid optical detection of nucleic acids.

All those aims have been accomplished with the present invention, as described herein and in the appended claims.

In view of the above-summarized drawbacks and/or problems affecting devices of the prior art, according to the present invention there is provided a molecular tool according to claim 1.

Particularly, in a first aspect the invention relates to a molecular tool comprising:
a substrate comprising or consisting of an optical material;
a first polymeric molecule selected from nucleic acids or nucleic acid analogues, coupled with the substrate; and
a second nucleic acid analogue coupled with the first polymeric molecule by means of nucleobase complementarity.

Another aspect of the present invention relates to use of the molecular tool of the invention for label-free detection of a target nucleic acid in a sample.

Another aspect of the present invention relates to a label-free method for detecting a target nucleic acid in a sample, the method comprising the steps of:
contacting a sample with a molecular tool of the invention under conditions allowing the coupling by means of nucleobase complementarity between the second nucleic acid analogue of the molecular tool and the target nucleic acid in the sample;
providing an electromagnetic radiation to the sample comprising the molecular tool under conditions allowing the interaction of the electromagnetic radiation with the optical material of the molecular tool; and
collecting and analysing an optical signal deriving from the optical material of the molecular tool, wherein said optical signal is indicative of the coupling between the second nucleic acid analogue and the target nucleic acid in the sample.

Another aspect of the present invention provides a kit comprising the molecular tool of the invention, at least one container and instructions for use.

Another aspect of the present invention provides a system for the detection of a target nucleic acid sequence in a sample, comprising the molecular tool of the invention, a source of electromagnetic radiation, and an electromagnetic radiation detector for collecting and analysing the optical signal deriving from the optical material of the molecular tool.

Described herein are molecular tools and methods for the optical detection of oligonucleotide binding events for diagnostic, point-of-care, drug screening applications and the like, for example, a robust and customizable system to detect specific DNA and RNA oligonucleotides, using a carbon nanotube optical signal. This optically based detection scheme is useful, e.g., for detecting circulating oligonucleotides that have diagnostic and prognostic value for cancer, metabolic disease, organ rejection, foetal health, and infectious disease. Potential targets include cell-free tumour DNA, circulating mRNA, and circulating microRNA (miRNA). Because this platform is compatible with biofluids, the platform provides, in various embodiments, purification-free, point-of-care diagnostics.

In a non-limiting, exemplary embodiment, the molecular tool and method of the invention combine the advantages of SWCNT-based sensors with those of synthetic biology oligomers, specifically PNA, and show the use of PNA-DNA-SWCNT hybrids to create a platform for the label-free detection of miRNA. Accordingly, with the molecular tool and method of the invention it is possible to form stable PNA-DNA-SWCNTs, which demonstrate optically distinct spectra compared to their DNA-SWCNT counterparts. In addition, the novel PNA-DNA-SWCNT sensors demonstrate rapid miRNA detection capabilities without a need for additional surfactant molecules during the detection process, making the sensors more applicable for in vivo sensing applications. It was shown that the length of the PNA-DNA hybrid section effects the performance of the sensor, indicating that the mechanism of response is not simply non-specific adsorption of the PNA/miRNA onto the DNA-SWCNT surface.

Furthermore, the modular approach for designing PNA-DNA-SWCNT sensors exemplarily presented in the present disclosure provides a proof-of-concept for a platform that can be readily adapted for a variety of different miRNA sequences.

Further embodiments of the present invention are defined by the appended claims.

The above and other objects, features and advantages of the herein presented subject-matter will become more apparent from a study of the following description with reference to the attached figures showing some preferred aspects of said subject-matter.

### BRIEF DESCRIPTION OF DRAWINGS

FIG. 1 shows detection of miRNA using PNA-DNA-SWCNT hybrids. (A) Schematic of PNA-DNA8bp-rev configuration used to create the sensor for miRNA detection. The DNA wrapping sequence consisted of a (GT)10 anchor and eight bases designed to hybridize with the PNA sequence. (B) PLE maps of the original DNA8bp-rev-SWCNT solution (left), the DNA8bp-rev-SWCNT solution mixed with PNA202 (PNA-DNA-SWCNT, center), and the PNA-DNA8bp-rev-SWCNT solution mixed with complementary miRNA (miR202, right). The predominate chiralities are labelled in white. All fluorescence intensities were normalized to the maximum intensity in each plot (final concentration: PNA=9.5 μM, miRNA=4.8 μM). (C) Spectra extracted from PLE maps to compare the intensity and wavelength positions of the DNA8bp-rev-SWCNTs (green), the PNA-DNA8bp-rev-SWCNTs (orange), and the PNA-DNA8bp-rev-SWCNTs following the addition of complementary miRNA (blue). The wavelength of the excitation laser used is indicated in the lower right corner (final concentration: PNA=9.5 μM, miRNA=4.8 μM). Comparison of the chirality dependent wavelength shifts following addition of miRNA (orange), PNA (green), and PNA with complementary miRNA (red) (final concentration: PNA=9 μM, miRNA=1 μM). All wavelength shifts were calculated versus the wavelength positions for DNA8bp-rev-SWCNTs. For DNA8bp-rev-SWCNTs, error bars represent 1æ standard error (n=5 technical replicates). For all other samples error bars represent the error on the fit of the peak (3æ from one technical replicate). Fluorescence wavelength responses of PNA-DNA8bp-rev-SWCNTs following the addition of complementary and non-complementary miRNA (final concentration: PNA=9 μM, miRNA=1 μM). Complementary miRNA was added to PNA202-, PNA195-, PNA184-, and PNA155-DNA8bp-rev-SWCNTs. Non-complementary miRNA was added to PNA10b-DNA8bp-rev-SWCNTs. All wavelength shifts were calculated versus the wavelength position of each chirality for the PNA-DNA8bp-rev-SWCNTs prior to miRNA addition. Red indicates a red-shift in the wavelength position of the chirality peak, and blue represents a blue-shift in the wavelength position.

FIG. 2 shows PLE maps of the PNA-DNA8bp-rev-SWCNT solutions (PNA195, PNA184, and PNA155) before (top) and after (bottom) addition of complementary miRNA (final concentration: PNA=9.5 μM, miRNA=4.8 μM). The predominate chiralities are labelled in white. All fluorescence intensities were normalized to the maximum intensity in each plot.

FIG. 3 shows spectra extracted from PLE maps to compare the intensity and wavelength positions of the DNA8bp-rev-SWCNTs (green), the PNA-DNA8bp-rev-SWCNTs (orange), and the PNA-DNA8bp-rev-SWCNTs following the addition of complementary miRNA (blue) for hybrids formed using PNA155 (left), PNA184 (center), or PNA195 (right) (final concentration: PNA=9.5 μM, miRNA=4.8 μM). The wavelength of the excitation laser used is indicated in the lower right corner.

FIG. 4 shows fluorescence wavelength shift of the (7,5), (7,6), (10,2), (9,4), and (8,6) peaks following the addition of PNA, miRNA, or PNA+miRNA to DNA8bp-rev-SWCNTs (final concentration: PNA=9 μM, miRNA=1 μM). All wavelength shifts were calculated versus the peak positions for DNA8bp-rev-SWCNTs. Red indicates a redshift in the wavelength position of the chirality peak and blue represents a blueshift in the wavelength position.

FIG. 5 shows comparison of the chirality dependent wavelength shifts (left) and intensity changes (right) for DNA8bp-rev-SWCNTs following addition of miRNA (orange), PNA (green), and PNA with miRNA (red) (final concentration: PNA=9 μM, miRNA=1 μM). All wavelength shifts were calculated versus the wavelength positions for DNA8bp-rev-SWCNTs. For DNA8bp-rev-SWCNTs, error bars represent 1æ standard deviation (n=5 technical replicates). For all other samples error bars represent the error on the fit of the peak (3æ from one technical replicate). For PNA10b (top) non-complementary miRNA (miR92a) was added while for all other samples the respective complementary miRNA was added.

FIG. 6 shows schematic of the PNA-DNA-SWCNT configurations differing in length of the hybrid portion (8, 10, 15, or 20 bases) and directionality of the hybridization (anti-parallel (rev) or parallel). PNA hybridizes with DNA in a sequence-dependent manner, according to the Watson-Crick hydrogen bonding scheme [322]. However, contrary to DNA, PNA can bind to complementary nucleic acids in both parallel and anti-parallel orientation, although the later is preferred and results in a more stable complex [319, 322].

FIG. 7 shows dependency of the wavelength shifting response of PNA-DNA-SWCNTs on the length of the DNA hybrid portion. Comparison of the wavelength shift following addition of complementary (left) and non-complementary (right) miRNA to PNA-DNA-SWCNTs (final concentration: PNA=9 μM, miRNA=1 μM). PNA-DNA-SWCNTs were formed using different lengths for the hybrid portion in the DNA sequence (8, 10, 15, or 20 bases) as well as different directionalities for the hybridization (anti-parallel (rev) or parallel). All wavelength shifts were calculated versus the wavelength position of each chirality for the PNA-DNA-SWCNTs prior to miRNA addition. Red indicates a red-shift in the wavelength position of the chirality peak, and blue represents a blue-shift in the wavelength position.

FIG. 8 shows comparison of the chirality dependent wavelength shifts (left) and intensity changes (right) for DNA8bp-SWCNTs following addition of miRNA (orange), PNA (green), and PNA with miRNA (red) (final concentration: PNA=9 μM, miRNA=1 μM). All wavelength shifts were calculated versus the wavelength positions for DNA8bp-SWCNTs. For DNA8bp-SWCNTs, error bars represent 1æ standard deviation (n=5 technical replicates). For all PNA samples with and without miRNA, error bars represent 1æ standard deviation (n=2 technical replicates). For miRNA additions in the absence of PNA error bars represent the error on the fit of the peak (3æ from one technical replicate). For PNA10b (top) non-complementary miRNA (miR92a) was added while for all other samples the respective complementary miRNA was added.

FIG. 9 shows comparison of the chirality dependent wavelength shifts (left) and intensity changes (right) for DNA10 bp-SWCNTs following addition of miRNA (orange), PNA (green), and PNA with miRNA (red) (final concentration: PNA=9 μM, miRNA=1 μM). All wavelength shifts were calculated versus the wavelength positions for DNA10 bp-SWCNTs. For DNA10 bp-SWCNTs and miRNA additions in the absence of PNA, error bars represent 1æ standard deviation (n=2 technical replicates). For all other samples, error bars represent 1æ standard deviation (n=3 technical replicates). For PNA10b (top) non-complementary miRNA (miR92a) was added while for all other samples the respective complementary miRNA was added.

FIG. 10 shows comparison of the chirality dependent wavelength shifts (left) and intensity changes (right) for DNA15 bp-SWCNTs following addition of miRNA (orange), PNA (green), and PNA with miRNA (red) (final concentration: PNA=9 μM, miRNA=1 μM). All wavelength shifts were calculated versus the wavelength positions for DNA15 bp-SWCNTs. For DNA15 bp-SWCNTs and miRNA additions in the absence of PNA, error bars represent 1æ standard deviation (n=2 technical replicates). For all other samples, error bars represent 1æ standard deviation (n=3 technical replicates). For PNA10b (top) non-complementary miRNA (miR92a) was added while for all other samples the respective complementary miRNA was added.

FIG. 11 shows comparison of the chirality dependent wavelength shifts (left) and intensity changes (right) for DNA15 bp-SWCNTs following addition of miRNA (orange), PNA (green), and PNA with miRNA (red) (final concentration: PNA=9 μM, miRNA=1 μM). All wavelength shifts were calculated versus the wavelength positions for DNA20 bp-SWCNTs. For DNA20 bp-SWCNTs and miRNA additions in the absence of PNA, error bars represent 1æ standard deviation (n=2 technical replicates). For all other samples, error bars represent 1æ standard deviation (n=3 technical replicates). For PNA10b (top) non-complementary miRNA (miR92a) was added while for all other samples the respective complementary miRNA was added.

FIG. 12 shows dependency of the wavelength shifting response of PNA155, PNA184- and PNA195-DNA-SWCNTs on the length of the DNA hybrid portion. Comparison of the wavelength shift following addition of complementary miRNA to PNA-DNA-SWCNTs (final concentration: PNA=9 μM, miRNA=1 μM). PNA-DNA-SWCNTs were formed using different lengths for the hybrid portion in the DNA sequence (8, 10, 15, or 20 bases) as well as different directionalities for the hybidization (anti-parallel (rev) or parallel). All wavelength shifts were calculated versus the wavelength position of each chirality for the respective PNA-DNA-SWCNTs prior to miRNA addition. Red indicates a redshift in the wavelength position of the chirality peak, and blue represents a blueshift in the wavelength position.

FIG. 13 shows fluorescence wavelength shift response of PNA-DNA15 bp-SWCNTs to complementary and non-complementary miRNA (final concentration: PNA=9.4 μM, miRNA=5.7 μM). complementary miRNA was added to PNA202-, PNA195-, PNA184-, and PNA155-DNA15 bp-SWCNTs, as well as the non-complementary miR25 for comparison. Two different non-complementary miRNAs (miR25 and miR92a) were added to PNA10b-DNA15 bp-SWCNTs. All wavelength shifts were calculated versus the wavelength position of the chiralities for the respective PNA-DNA15 bp-SWCNT solution prior to miRNA addition. Red and blue indicate a red-shift or blue-shift in the wavelength position, respectively.

FIG. 14 shows comparison of the chirality dependent wavelength shifts (left) and intensity changes (right) for DNA15 bp-SWCNTs following addition of miRNA (orange), PNA (green), and PNA with either complementary (red) or non-complementary (purple) miRNA. Higher relative concentrations of miRNA were added to increase the ration of miRNA: PNA (final concentration: PNA=9.4 μM, miRNA=5.7 μM). Complementary miRNA was added to PNA202-, PNA195-, PNA184-, and PNA155-DNA15 bp-SWCNTs, as well as the non-complementary miR25 for comparison. Two different non-complementary miRNAs (miR25 and miR92a) were added to PNA10b-DNA15 bp-SWCNTs. All wavelength shifts were calculated versus the wavelength position of the chiralities for the respective DNA15 bp-SWCNT solution prior to any addition. For complementary miRNA additions error bars represent 1æ standard deviation (n=3 technical replicates), for all other samples error bars represent 1æ standard deviation (n=2 technical replicates).

FIG. 15 shows PLE maps of the PNA-15 bp-SWCNT solutions (PNA10b, PNA155, PNA184, PNA195, and PNA202) before (left) and after addition of complementary (center) and non-complementary (miR25, right) miRNA (final concentration: PNA=9.4 μM, miRNA=5.7 μM). For the PNA10b-DNA15 bp-SWCNT solution two non-complementary miRNAs were added (miR92a—center, miR25—right). The predominate chiralities are labelled in white. All fluorescence intensities were normalized to the maximum intensity in each plot.

FIG. 16 shows comparison of the normalized absorbance spectra for DNA15 bp-SWCNTs (red), PNA155-DNA15 bp-SWCNTs or PNA10b-DNA15 bp-SWCNTs before (green) and after the addition of either complementary or non-complementary miRNA (purple). Samples were incubated for 15 min following the addition of miRNA prior to acquiring the spectrum. PNA-DNA samples were incubated for 120 min prior to addition of the miRNA sequence (final concentrations: PNA=9.4 μM, miRNA=5.7 μM).

FIG. 17 shows comparison of the normalized absorbance spectra for DNA15 bp-SWCNTs (red), PNA155-DNA15 bp-SWCNTs (green), and PNA155-DNA15 bp-SWCNTs following the addition of either complementary (miR155, left) or non-complementary (miR25, right) miRNA (purple). Samples were incubated for 30 min following the addition of PNA prior to acquiring the spectrum. PNA-DNA samples used for miRNA detection were incubated for 2 h prior to addition of the miRNA sequence (final concentrations: PNA=9.4 μM, miRNA=5.7 μM).

FIG. 18 shows PLE maps of the PNA-10 bp-SWCNT solutions (PNA10b, PNA155, PNA184, PNA195, and PNA202) before (left) and after addition of complementary (center) and non-complementary (miR25, right) miRNA (final concentration: PNA=9.4 μM, miRNA=5.7 μM). For the PNA10b-DNA10 bp-SWCNT solution two non-complementary miRNAs were added (miR92a—center, miR25—right). The predominate chiralities are labelled in white. All fluorescence intensities were normalized to the maximum intensity in each plot.

FIG. 19 shows PLE maps of the PNA-20 bp-SWCNT solutions (PNA10b, PNA155, PNA184, PNA195, and PNA202) before (left) and after addition of complementary (center) and non-complementary (miR25, right) miRNA (final concentration: PNA=9.4 μM, miRNA=5.7 μM). For the PNA10b-DNA20 bp-SWCNT solution two non-complementary miRNAs were added (miR92a—center, miR25—right). The predominate chiralities are labelled in white. All fluorescence intensities were normalized to the maximum intensity in each plot.

FIG. 20 shows fluorescence wavelength shift response of PNA-DNA10 bp-SWCNTs and PNA-DNA20 bp-SWCNTs to complementary and non-complementary miRNA (final concentration: PNA=9.4 μM, miRNA=5.7 μM). complementary miRNA was added to PNA202-, PNA195-, PNA184-, and PNA155-DNA15 bp-SWCNTs. Non-complementary miRNAs (miR92a) were added to PNA10b-DNA15 bp-SWCNTs. All wavelength shifts were calculated versus the wavelength position of the chiralities for the respective PNA-DNA10 bp-SWCNT or PNA-DNA20 bp-SWCNT solution prior to miRNA addition. Red and blue indicate a redshift or blueshift in the wavelength position, respectively.

FIG. 21 shows a list of DNA sequences used to prepare the solutions of SWCNTs used in this study. All sequences consisted of a (GT)10-anchor sequence and 8-20 nucleotide for conjugation with the complementary PNA sequence. All suspensions were prepared using MeOH assisted surfactant exchange. Solutions were stored at 4±C to prevent aggregation. PNA was added to the mixtures immediately prior to measurement.

### DETAILED DESCRIPTION OF THE INVENTION

The subject-matter described in the following will be clarified by means of a description of those aspects which are depicted in the drawings. It is however to be understood that the scope of protection of the invention is not limited to those aspects described in the following and depicted in the drawings; to the contrary, the scope of protection of the invention is defined by the claims. Moreover, it is to be understood that the specific conditions or parameters described and/or shown in the following are not limiting of the scope of protection of the invention, and that the terminology used herein is for the purpose of describing particular aspects by way of example only and is not intended to be limiting.

Unless otherwise defined, technical and scientific terms used herein have the same meaning as commonly understood by one of ordinary skill in the art to which this invention belongs. Further, unless otherwise required by the context, singular terms shall include pluralities and plural terms shall include the singular. The methods and techniques of the present disclosure are generally performed according to conventional methods well known in the art and as described in various general and more specific references that are cited and discussed throughout the present specification unless otherwise indicated. Further, for the sake of clarity, the use of the term “about” is herein intended to encompass a variation of +/−10% of a given value.

Non-limiting aspects of the subject-matter of the present disclosure will be described by way of example with reference to the accompanying figures, which are schematic and are not intended to be drawn to scale. For purposes of clarity, not every component is labelled in every figure, nor is every component of each aspect of the invention shown where illustration is not necessary to allow those of ordinary skill in the art to understand the invention.

The following description will be better understood by means of the following definitions.

As used in the following and in the appended claims, the singular forms “a”, “an” and “the” include plural referents unless the context clearly dictates otherwise. Also, the use of “or” means “and/or” unless stated otherwise. Similarly, “comprise”, “comprises”, “comprising”, “include”, “includes” and “including” are interchangeable and not intended to be limiting. It is to be further understood that where for the description of various embodiments use is made of the term “comprising”, those skilled in the art will understand that in some specific instances, an embodiment can be alternatively described using language “consisting essentially of” or “consisting of.”

As used herein, an “optical material” is a material whose function is to alter or control electromagnetic radiation in the ultraviolet (UV), visible, or infrared (IR) spectral regions. Such materials may be fabricated into optical elements such as lenses, mirrors, windows, prisms, polarizers, detectors, and modulators, at macroscopic as well as microscopic scale, and they can be used to refract, reflect, transmit, disperse, partially absorb, polarize, detect, and/or transform light. At the microscopic level, atoms and their electronic configurations in the material interact with the electromagnetic radiation (photons) to determine its macroscopic optical properties such as transmission and refraction. Organic synthetic polymers are emerging as key materials in this field, and have an advantage over inorganic materials because they can be designed and synthesized into compositions and architectures not possible with crystals and glasses. Exemplary optical materials suitable in the frame of the present disclosure include Carbon Nanotubes (CNTs), Single Wall Carbon Nanotubes (SWCNTs), graphene, quantum dots, graphene quantum dots, plasmonically enhanced nanoparticles (for example, plasmonically coupled single-walled carbon nanotubes), fluorophores (such as dyes and fluorescent proteins), either in bulk or in a dispersed form, depending on the needs and circumstances. For instance, a thin film or membrane can be used as a support for immobilization of SWCNTs, or SWCNTs can be used in molecular dispersion in a liquid medium. In some preferred embodiments, the optical material is selected from the group comprising Carbon Nanotubes (CNTs), Single Wall Carbon Nanotubes (SWCNTs), quantum dots, graphene quantum dots, plasmonically enhanced nanoparticles and graphene.

The term “nucleotide” refers to a molecule that contains a nitrogen-containing heterocyclic base (also referred to as “nucleobase”), a sugar or a modified sugar and one or more phosphate groups. A nucleotide refers to a ribonucleotide, deoxynucleotide or a modified form of either type of nucleotide. For example, in some embodiments, a nucleotide can be a deoxynucleotide triphosphate (dNTP). The term “non-natural nucleotide” as used herein refers to a nucleotide that obeys Watson-Crick base pairing but has a modification that can be detected. By way of example, but not limitation, such a modification can be a functional group attached to the nucleobase such as a methyl group on methylcytosine.

According to the present disclosure, the terms “nucleic acid”, “nucleic acid molecule”, “nucleic acid sequence”, “nucleic acid fragment”, “oligonucleotide” and “polynucleotide” are used interchangeably and refer to biopolymers that are made from nucleotides as monomer units. The nucleotide monomers link up to form a linear sequence of the nucleic acid polymer. Nucleic acids encompassed by the present disclosure can include deoxyribonucleic acid (DNA), genomic DNA, ribonucleic acid (RNA) such as mRNA, siRNA, shRNA, miRNA and the like, and cDNA.

The term “nucleic acid molecule” usually refers to a molecule of at least 10 bases in length, unless otherwise specified. The term includes single and double stranded forms of DNA. In addition, a polynucleotide may include either or both naturally occurring and modified nucleotides linked together by naturally occurring and/or non-naturally occurring nucleotide linkages.

The nucleic acid molecules may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.)

As indicated above, the nucleic acids of the present disclosure can also include synthetic variants of DNA or RNA. “Synthetic variants” or “nucleic acid analogues” encompass nucleic acids incorporating known analogues of natural nucleotides/nucleobases that e.g. can hybridize to nucleic acids in a manner similar to naturally occurring nucleotides. Exemplary nucleic acid analogues include peptide nucleic acids (PNAs), phosphorothioate DNA, locked nucleic acids, and the like. Modified or synthetic nucleobases and analogues can include, but are not limited to, 5-Br-UTP, 5-Br-dUTP, 5-F-UTP, 5-F-dUTP, 5-propynyl dCTP, 5-propynyl-dUTP, diaminopurine, S2T, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N 6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-Dmannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Persons of ordinary skill in the art can readily determine what base pairings for each modified nucleobase are deemed a base-pair match versus a base-pair mismatch. Preferred nucleic acid analogues according to the present disclosure include so-called “Xeno nucleic acids” (XNA) such as glycerol nucleic acid (GNA), threose nucleic acid (TNA), locked nucleic acid (LNA), peptide nucleic acids (PNAs) cyclohexenyl nucleic acid (CeNA), hexose nucleic acid (HNA) or other synthetic polymers with nucleotide side chains, or any combination thereof. For the sake of easiness, peptide nucleic acids (PNAs), artificially synthesized polymer similar to DNA or RNA, are also included into the definition of nucleic acids according to the invention. PNA has an uncharged backbone which enables stronger DNA hybridization due to the absence of electrostatic repulsion, can hybridize with DNA virtually independently of the salt concentration, and has increased specificity compared to DNA or RNA.

The term “nucleic acid” or “nucleic acid molecule” also includes any topological conformation, including single-stranded, double-stranded, partially duplexed, triplexed, hairpinned, circular and padlocked conformations. Also included are synthetic molecules that mimic polynucleotides in their ability to bind to a designated sequence via hydrogen bonding and other chemical interactions. Such molecules are known in the art and include, for example, those in which peptide bonds substitute for phosphate bonds in the backbone of the molecule.

Antisense nucleic acid compositions, or vectors that drive expression of an antisense nucleic acid, are administered to downregulate transcription and/or translation of a gene product in circumstances in which e.g. excessive production, or production of aberrant protein, is the pathophysiologic basis of disease. Antisense compositions useful in therapy can have a sequence that is complementary to coding or to noncoding regions of a gene or gene product deriving therefrom. For example, oligonucleotides derived from the transcription initiation site, e.g., between positions −10 and +10 from the start site, are preferred. Catalytic antisense compositions, such as ribozymes, that are capable of sequence-specific hybridization to gene transcripts, are also useful in therapy.

The terms “microRNA”, “miRNA”, and “MIR” are interchangeable and refer to endogenous or artificial non-coding RNAs that are capable of regulating gene expression. It is believed that miRNAs function via RNA interference.

The terms “siRNA” and “short interfering RNA” are interchangeable and refer to single-stranded or double-stranded RNA molecules that are capable of inducing RNA interference. siRNA molecules typically have a duplex region that is between 18 and 30 base pairs in length.

The terms “piRNA” and “Piwi-interacting RNA” are interchangeable and refer to a class of small RNAs involved in gene silencing. PiRNA molecules typically are between 26 and 31 nucleotides in length.

The terms “snRNA” and “small nuclear RNA” are interchangeable and refer to a class of small RNAs involved in a variety of processes including RNA splicing and regulation of transcription factors. The subclass of small nucleolar RNAs (snoRNAs) is also included. The term is also intended to include artificial snRNAs, such as antisense derivatives of snRNAs comprising antisense sequences directed against one or more nucleic acid sequence of the invention, or to a variant or fragment thereof.

The term “shRNA” as used herein refers to a nucleic acid molecule comprising at least two complementary portions hybridized or capable of hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex.

“Single-walled carbon nanotubes” or “SWCNTs” are rolled sheets of graphene with nanometer-sized diameters. SWCNTs are defined by their chirality. The sheets that make up the SWCNTs are rolled at specific and discrete, i.e., “chiral” angles. This rolling angle in combination with the nanotube radius determines the nanotube's properties. SWCNTs of different chiralities have different electronical properties. These electronic properties are correlated with respective differences in optical properties. Thus, individually-dispersed semiconducting SWCNTs exhibit ideal qualities as optical biomedical sensors.

Semiconducting SWCNTs are fluorescent in the near-infrared (NIR, 900-1600 nm) due to their electronic band-gap between valence and conduction band. The semiconducting forms of SWNTs, when dispersed by surfactants in aqueous solution, can display distinctive near-infrared (IR) photoluminescence arising from their electronic band gap. The band-gap energy is sensitive to the local dielectric environment around the SWNT, and this property can be exploited in chemical sensing. Among the molecules that can bind to the surface of SWNTs is DNA, which adsorbs as a double-stranded (ds) complex.

The term “subject” or “patient” as used herein refers to animals, including mammals, birds, fish, invertebrates, insects and so forth. Mammals contemplated by the present invention include humans, primates, domesticated animals such as cattle, sheep, pigs, horses, laboratory rodents and the like.

The term “tumour” or “cancer”, as used herein, refers to all neoplastic cell growth and proliferation, whether malignant or benign, and all pre-cancerous and cancerous cells and tissues. Additionally or alternatively, the terms refer to the presence of cells possessing characteristics typical of cancer-causing cells, such as uncontrolled proliferation, immortality, metastatic potential, rapid growth and proliferation rate, and certain characteristic morphological features. The term “cancerous” refer to or describe the physiological condition in mammals that is typically characterized by unregulated cell growth. Examples of cancer include, but are not limited to, blood cancer, breast cancer, ovarian cancer, colon cancer, lung cancer, prostate cancer, hepatocellular cancer, gastric cancer, pancreatic cancer, cervical cancer, liver cancer, bladder cancer, cancer of the urinary tract, thyroid cancer, renal cancer, carcinoma, melanoma, and brain cancer.

As used herein, “treatment”, “treating” and the like generally refers to any act intended to ameliorate the health status of subjects such as an animal, particularly a mammal, more particularly a human, such as therapy, prevention, prophylaxis and retardation of the disease, and includes: (a) inhibiting the disease, i.e., arresting its development; or (b) relieving the disease, i.e., causing regression of the disease and/or its symptoms or conditions such as improvement or remediation of damage. In certain aspects, such term refers to the amelioration or eradication of a disease or symptoms associated with a disease. In other aspects, this term refers to minimizing the spread or worsening of the disease resulting from the administration of one or more therapeutic agents to a subject with such a disease. The term “prevention” or “preventing” relates to hampering, blocking or avoid a disease from occurring in a subject which may be, for any reason, predisposed to the disease but has not yet been diagnosed as having it for example based on familial history, health status or age.

Described herein are molecular tools and methods for the optical detection of oligonucleotide binding events for diagnostic, point-of-care, drug screening, and theranostic applications, for example, a robust and customizable system to detect specific DNA and RNA oligonucleotides, using an optical signal. This optically based detection scheme is useful, e.g., for detecting circulating oligonucleotides that have diagnostic and prognostic value for cancer, metabolic disease, organ rejection, fetal health, and infectious disease. Potential targets include cell-free tumor DNA, circulating mRNA, and circulating microRNA (miRNA). Because this platform is compatible with biofluids, the platform provides, in various embodiments, purification-free, point-of-care diagnostics.

An aspect of the present invention relates to a molecular tool comprising:
a substrate comprising or consisting of an optical material;
a first polymeric molecule, selected from nucleic acids or nucleic acid analogues, coupled with the substrate; and
a second nucleic acid analogue coupled with the first polymeric molecule by means of nucleobase complementarity.

In some embodiments of the molecular tool of the invention, said optical material is selected from Carbon Nanotubes (CNTs), Single Wall Carbon Nanotubes (SWCNTs), graphene, quantum dots, graphene quantum dots, plasmonically enhanced nanoparticles or fluorophores. In a preferred embodiment of the molecular tool of the invention, said optical material is Single Wall Carbon Nanotubes (SWCNTs). In another preferred embodiment of the molecular tool of the invention, said optical material is a stable dispersion of isolated Single Wall Carbon Nanotubes.

In other embodiments of the molecular tool of the invention, said nucleic acids are deoxyribonucleic acids (DNA) or ribonucleic acids (RNA). In other embodiments of the molecular tool of the invention, said nucleic acid analogues are selected from glycerol nucleic acid (GNA), threose nucleic acid (TNA), locked nucleic acid (LNA), peptide nucleic acids (PNAs) cyclohexenyl nucleic acid (CeNA), or hexose nucleic acid (HNA).

In the molecular tool of the invention, said second nucleic acid analogue is coupled with said first polymeric molecule by partial overlap. Further, in the molecular tool of the invention, the first polymeric molecule and the second nucleic acid analogue comprise preferably at least a single-stranded portion for coupling by means of nucleobase complementarity. Further, in the molecular tool of the invention, the second nucleic acid analogue is configured to bind a complimentary target nucleic acid or comprises a domain capable of binding a target nucleic acid.

An embodiment of the present invention provides the molecular tool comprising:
a substrate comprising or consisting of an optical material selected from Carbon Nanotubes (CNTs), Single Wall Carbon Nanotubes (SWCNTs), graphene, quantum dots, graphene quantum dots, plasmonically enhanced nanoparticles or fluorophores; preferably an optical material is Single Wall Carbon Nanotubes (SWCNTs) or a stable dispersion of isolated Single Wall Carbon Nanotubes;
a first polymeric molecule, selected from nucleic acids or nucleic acid analogues, coupled with the substrate; and
a second nucleic acid analogue coupled with the first polymeric molecule by means of nucleobase complementarity,
   wherein
said nucleic acids are deoxyribonucleic acids (DNA) or ribonucleic acids (RNA);
said nucleic acid analogues are selected from glycerol nucleic acid (GNA), threose nucleic acid (TNA), locked nucleic acid (LNA), peptide nucleic acids (PNAs) cyclohexenyl nucleic acid (CeNA), or hexose nucleic acid (HNA);
said second nucleic acid analogue is coupled with said first polymeric molecule by partial overlap;
said the first polymeric molecule and said second nucleic acid analogue comprise preferably at least a single-stranded portion for coupling by means of nucleobase complementarity; and
said second nucleic acid analogue is configured to bind the target nucleic acid or comprises a domain capable of binding the target nucleic acid.

A molecular tool according to the invention is, or comprises, an optical sensor engineered for real-time optical quantification of hybridization events of microRNA and other oligonucleotides. Without being bounded to any theory, it is hypothesized that the mechanism of the sensors arise from competitive effects between displacement of both oligonucleotide charge groups and water from the surface of a substrate comprising or consisting of an optical material, which result in a solvatochromism-like response.

In one embodiment according to the invention, said optical material comprises or consists of Single Wall Carbon Nanotubes (SWCNTs). In a preferred embodiments, said optical material comprises or consists of a stable dispersion of isolated Single Wall Carbon Nanotubes. Individually-dispersed semiconducting Single Wall Carbon Nanotubes exhibit ideal qualities as optical biomedical sensors. SWCNTs are fluorescent in the near-infrared, a wavelength range penetrant to tissue, raising the possibility of implantable sensors. Additionally, SWCNTs do not photobleach due to their excitonic nature of fluorescence. The emission wavelength and intensity is exquisitely sensitive to the immediate SWCNT environment, allowing changes at the surface to be transduced in an optical signal. Sensitivity to some analytes has been measured at the single-molecule level. It has been shown that single-strand DNA has an affinity for the nanotube surface and can be used as a dispersant to prepare optically active, single nanotube dispersions. Additionally, DNA-DNA hybridization between nanotube-associated DNA and free single-strand DNA in solution can mediate a solvatochromic shift in the nanotube emission.

In every embodiment, a distinguishing feature is that the first polymeric molecule, selected from nucleic acids or nucleic acid analogues, coupled with the substrate includes both a substrate-binding, such as a nanotube-binding, domain and a second domain that hybridizes (couples) with a second nucleic acid analogue by means of nucleobase complementarity. The substrate-binding domain can functionally couple the first polymeric molecule to the substrate by means covalent or non-covalent bindings, such as for example through of Van der Waals interactions or pi-pi stacking. In embodiments where SWCNTs are used, the first polymeric molecule can be coupled through its first coupling domain via wrapping of this latter around SWCNTs.

Preferably, and within the scope of the invention, said second nucleic acid analogue is coupled with said first polymeric molecule by partial overlap, and particularly via nucleobase complementarity. In preferred embodiments, said second nucleic acid analogue and, whenever present, said first nucleic acid analogue, is/are PNAs. PNAs show the advantages of having an uncharged backbone which enables stronger DNA hybridization due to the absence of electrostatic repulsion, can hybridize with DNA virtually independently of the salt concentration in a test sample, and has increased specificity compared to DNA or RNA. In other preferred embodiments, said first polymeric molecule is DNA.

Another aspect of the present disclosure relates to the use of the molecular tool of the invention for label-free detection of a target nucleic acid in a sample.

Another aspect of the present invention relates to a label-free method for detecting a target nucleic acid in a sample, the method comprising the steps of:
contacting a sample with a molecular tool according to the invention under conditions allowing the coupling by means of nucleobase complementarity between the second nucleic acid analogue of the molecular tool and a target nucleic acid in the sample;
providing an electromagnetic radiation to the sample comprising the molecular tool under conditions allowing the interaction of the electromagnetic radiation with the optical material of the molecular tool; and
collecting and analysing an optical signal deriving from the optical material of the molecular tool, wherein said optical signal is indicative of the coupling between the second nucleic acid analogue and the target nucleic acid in the sample.

In an embodiment of the method of the invention, said target nucleic acid is cell-free tumour DNA, circulating mRNA, or circulating microRNA (miRNA); preferably said target nucleic acid in the sample is a miRNA.

In another embodiment of the method of the invention, said sample is a biological sample selected from blood, plasma, saliva, urine, biopsies, organs, tissues or cell samples.

In a further embodiment of the method of the invention, said sample is a native, unprocessed biological sample.

In a further embodiment of the method of the invention, said electromagnetic radiation is selected from visible light, UV light or IR light.

In a further embodiment of the method of the invention, said optical signal is selected from fluorescence or fluorescence wavelength shift.

In a further embodiment of the method of the invention, the step of collecting and analysing an optical signal comprises performing a photoluminescence excitation/emission spectroscopy analysis in the near IR spectrum.

In advantageous embodiment, the method is performed in the absence of any surfactant. Indeed, surfactants (especially surfactants concentration) often provide problems in vivo since surfactants are often soluble in aqueous solutions, including bio fluids, and would readily solubilize/become diluted over time as fluids circulate in the body. Further, there is a problem in terms of reproducibility and calibration of sensors, since the performance now become dependent on surfactant concentration. Also, surfactants can have toxic effects (a problem that is exacerbated if there is a need to keep pumping in surfactants onto a localized sensor just to maintain surfactant concentrations. Surfactants can also have denaturing effects on proteins and biomolecules. In general, surfactants also have major effects on altering the bioenvironment, and these effects are concentration dependent. For example, they can competitively solubilize not only nanotubes, but also proteins. If surfactants have a preference for proteins, then the needed amount to be added to solubilize the nanotubes will be higher for solutions that have more proteins in them.

The method of the invention is for sensing and/or characterising a target nucleic acid. The method is for sensing and/or characterising at least one target nucleic acid. The method may concern sensing and/or characterising two or more target nucleic acids.

In one embodiment, the method of the invention can be used to detect any number of target nucleic acids, such as 1, 2, 5, 10, 15, 20, 30, 40, 50, 100 or more target nucleic acids in a same or different samples. The method of the invention may concern detecting two or more target nucleic acids of the same type, such as two or more oligo- or polynucleotides or miRNA. When two or more target nucleic acids should be detected in the same sample or different samples, the method uses two or more different molecular tools, each of these molecular tools being specific for one target nucleic acid in a sample, i.e each molecular tool comprising a second nucleic acid analogue configured to bind one specific target nucleic acid or a second nucleic acid analogue comprises a domain capable of binding the one specific target nucleic acid.

The target nucleic acid can be secreted from cells. Alternatively, the target nucleic acid can be a nucleic acid substrate that is present inside cells such that the substrate must be extracted from the cells before the invention can be carried out.

The target nucleic acid can be of any length. For example, the polynucleotide can be at least 10, at least 50, at least 100, at least 150, at least 200, at least 250, at least 300, at least 400 or at least 500 nucleotide pairs in length. The polynucleotide can be 1000 or more nucleotide pairs, 5000 or more nucleotide pairs in length or 100000 or more nucleotide pairs in length. In some embodiments, the target nucleic acid is cell-free tumour DNA, circulating mRNA or circulating microRNA (miRNA). In other embodiments, the target nucleic acid in a sample is a miRNA.

The target nucleic acid, such as a target polynucleotide, is present in any suitable sample. The invention is typically carried out on a sample that is known to contain or suspected to contain the target nucleic acid, such as the target polynucleotide. Alternatively, the invention may be carried out on a sample to confirm the identity of one or more target nucleic acids, such as one or more target polynucleotides, whose presence in the sample is known or expected. Preferably, the sample is in a liquid form.

In embodiments of the invention, the sample may be a biological sample. In this context, the term “sample” means any sample containing cells and/or nucleic acids derived and/or obtained from a subject. Examples of such samples include fluids such as blood, plasma, saliva, and urine as well as biopsies, organs, tissues or cell samples.

The invention may be carried out in vitro on a sample obtained from or extracted from any organism or microorganism. The organism or microorganism is typically archaean, prokaryotic or eukaryotic and typically belongs to one the five kingdoms: plantae, animalia, fungi, monera and protista. The invention may be carried out in vitro on a sample obtained from or extracted from any virus. The sample is preferably a fluid sample. The sample typically comprises a body fluid of a patient. The sample may be without limitation urine, lymph, saliva, mucus, amniotic fluid, blood, plasma or serum. Typically, the sample is human in origin, but alternatively it may be from another mammal animal such as from commercially farmed animals such as horses, cattle, sheep or pigs or may alternatively be pets such as cats or dogs. Advantageously, and within the scope of the invention, said sample is a native, unprocessed biological sample.

In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

However, in embodiments of the invention, the sample may be a non-biological sample. The non-biological sample is preferably a fluid sample. Examples of a non-biological sample include surgical fluids, water such as drinking water, sea water or river water, and reagents for laboratory tests.

The sample may be minimally processed prior to being assayed, for example by centrifugation or by passage through a membrane that filters out unwanted molecules or cells, such as red blood cells. The sample may be measured immediately upon being taken. The sample may also be typically stored prior to assay, preferably below −70° C.

The method according to the invention foresees exposing the sample to excitation electromagnetic radiation (excitation EMR) under conditions allowing the interaction of the electromagnetic radiation with the optical material of the molecular tool, in particular to produce an optical signal such as emission of electromagnetic radiation (emission EMR) by said optical material. The optical signal deriving from the optical material is detected and analysed to identify the presence of the species having a target nucleotide sequence in the sample, thereby indicating the coupling between the nucleic acid analogue in the molecular tool of the invention and said target nucleic acid in the sample. This step is based at least in part on the detection of an emission EMR.

In certain embodiments, the method comprises detecting a wavelength shift (e.g., a blueshift or a redshift) in the emission EMR and/or an intensity shift (e.g., amplitude shift) or other changes in the spectral characteristics of the emission EMR or non-emission EMR changes, thereby identifying the presence of the species having the target nucleotide sequence in the test sample.

In certain embodiments, the other changes in the spectral characteristics of the emission EMR include ratiometric intensity changes (e.g., relative changes of one nanotube chirality intensity versus another), changes in full-width half-max (e.g., a measure of the “thickness” of the spectral peak), changes in exciton energy transfer (a unique spectral signature from energy exchange between nanotubes in close-contact), and combinations thereof.

In certain embodiments, the non-emission EMR changes include changes in light absorbance (such as bleaching), blueshift or redshift in the excitation EMR, changes in dynamic light scattering (sample bundling), visible flocculation (aggregation) of nanotubes in sample, and combinations thereof.

In certain embodiments, the method comprises detecting an intensity shift between an emission center wavelength (e.g., a peak) of the sample and an emission center wavelength (e.g., a peak) of a reference sample, wherein the reference sample is devoid of the species having the target nucleotide sequence.

In certain embodiments, the excitation EMR has a wavelength between 100 nm and 3000 nm, 200 nm and 2000 nm, between 300 and 1500 nm, or between 500 and 1000 nm. In certain embodiments, the emission EMR has a wavelength between 300 nm and 3000 nm, between 400 and 2000 nm, between 500 and 1500 nm, between 600 nm and 1400 nm, or between 700 and 1350 nm. In certain embodiments, the emission wavelength shift is between 1 nm and 100 nm, between 2 nm and 100 nm, between 3 and 50 nm, or between 4 and 20 nm.

In certain embodiments, the wavelength shift is a blue shift.

In certain embodiments, the species having the target nucleotide sequence is microRNA.

Target conditions and diseases that can be diagnosed, treated and/or prevented using the molecular tools and methods described herein include cancers, metabolic diseases, foetal health conditions, kidney diseases, organ rejections, hereditary diseases, nervous diseases, obesity and infectious diseases.

Advantageously, the method of the invention does not require special analytical work and equipment (such as PCR) and can be performed with fewer steps than other methods that require multiple incubation, bioconjugation, and washing steps.

In certain embodiments, the methods described herein can be used for diagnostic or therapeutic purposes to diagnose, prevent, or treat any condition or disease characterized by or associated with a target nucleic acid as described herein.

Sources of excitation EMR can be any such source known in the art, e.g., a laser, a light emitting diode, or a lamp. Detectors of emission EMR can be any such detector known in the art, e.g., a fluorometer. In certain embodiments, the method comprises detecting a wavelength shift (e.g., a blue or red shift) in the emission EMR and/or an intensity shift (e.g., amplitude shift), or other changes in the spectral characteristics of in the emission EMR, thereby identifying the presence of the species having the target nucleotide sequence in the test sample.

Another aspect of the present invention relates to a kit comprising as least one molecular tool of the invention, at least one container and instructions for use.

In some embodiments, the at least one container is selected from an ampule, a vial, a cartridge, a reservoir, a pre-filled syringe, a pre-filled cartridge, a pre-filled petri dish, or a pre-filled reservoir. The at least one container can be adapted to receive a sample to be analysed.

When two or more target nucleic acids should be detected in the same sample or different samples, the kit comprises two or more different molecular tools, each of these molecular tools being specific for one target nucleic acid in a sample, i.e each molecular tool comprising a second nucleic acid analogue configured to bind one specific target nucleic acid or a second nucleic acid analogue comprises a domain capable of binding the one specific target nucleic acid.

Kits of the invention may include other components required to conduct the methods of the present invention, such as buffers and/or diluents. The kits may comprise one or more means for obtaining a sample from a subject. The kits typically include containers for housing the various components and/or instructions for using the kit components in the methods of the invention.

Kits of the invention may further comprise a carrier, package or container that is compartmentalized to receive one or more containers such as vials, tubes, and the like, each of the container(s) comprising one of the separate elements to be used in the methods of the invention. The kits of the invention will typically comprise the container comprising the elements described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for either in vivo or in vitro use. Directions and or other information can also be included on an insert which is included with the kits. The kits preferably comprise means for handling and/or processing a sample, such as a biological sample (blood sample blood, plasma, saliva, urine, biopsies, organs, tissues or cell samples).

Another aspect of the present invention provides a system for the detection of a target nucleic acid sequence in a sample, comprising the molecular tool of the invention, a source of electromagnetic radiation, and an electromagnetic radiation detector for collecting and analysing the optical signal deriving from the optical material of the molecular tool. In some embodiments of the system of the invention, said target nucleic acid is cell-free tumour DNA, circulating mRNA, or circulating microRNA (miRNA); preferably said target nucleic acid in the sample is a miRNA. In other embodiments of the system of the invention, said sample is a biological sample selected from blood, plasma, saliva, urine, biopsies, organs, tissues or cell samples. Preferably, said sample is a native, unprocessed biological sample. In other embodiments of the system of the invention, said electromagnetic radiation is selected from visible light, UV light or IR light. In other embodiments of the system of the invention, said optical signal is selected from fluorescence or fluorescence wavelength shift. In another embodiment of the system of the invention, the collecting and analysing an optical signal comprises performing a photoluminescence excitation/emission spectroscopy analysis in the near IR spectrum.

### Examples

In the following, exemplary non-limiting embodiments of the molecular tools and methods according to the present disclosure are described.

A modular assembly for PNA-DNA-SWCNTs, where DNA scaffolds were used to attach PNA to the SWCNT surface, was firstly designed to obtain stable dispersions of isolated SWCNTs. Initial experiments used a DNA sequence containing a (GT)₁₀ anchor section and an eight base long hybrid section, whose sequence was reverse complementary to the hybrid portion for all PNA sequences (5′-GTCTCTTC-3′), hence forth referred to as DNA_{8bp-rev}. DNA_{8bp-rev} was used to prepare nanotube dispersions via MeOH-assisted surfactant exchange.

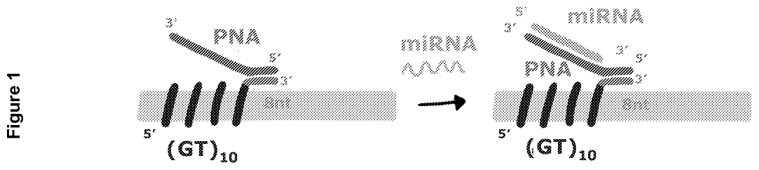

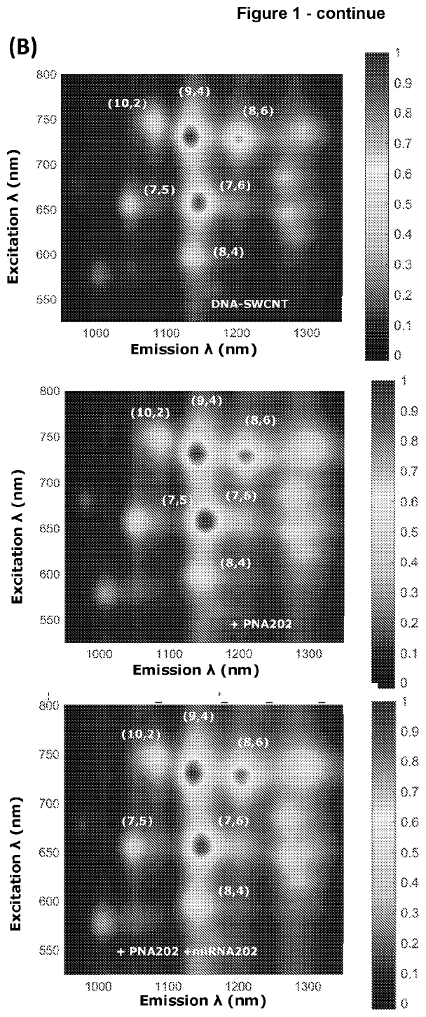

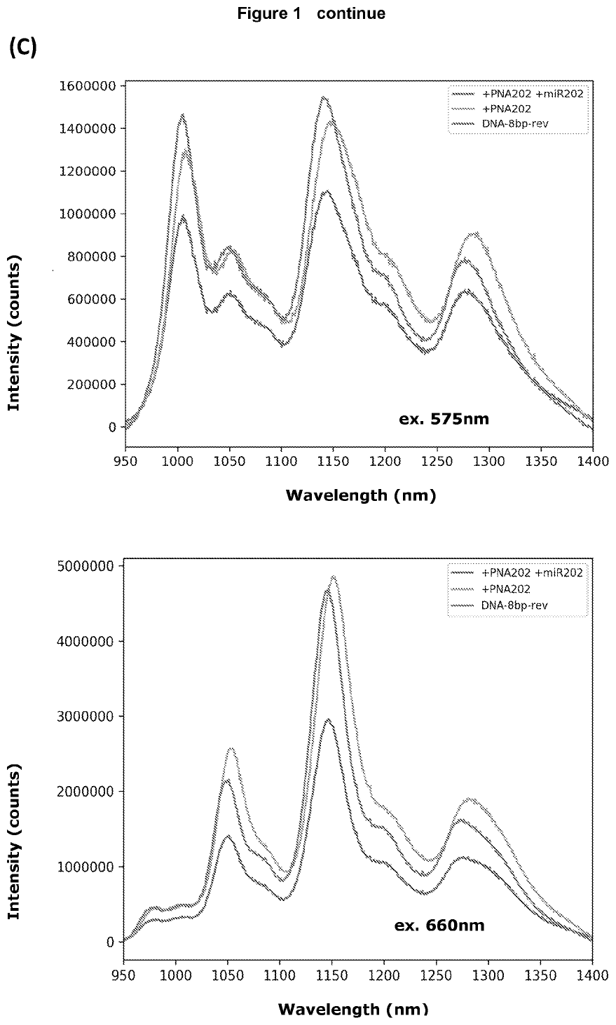

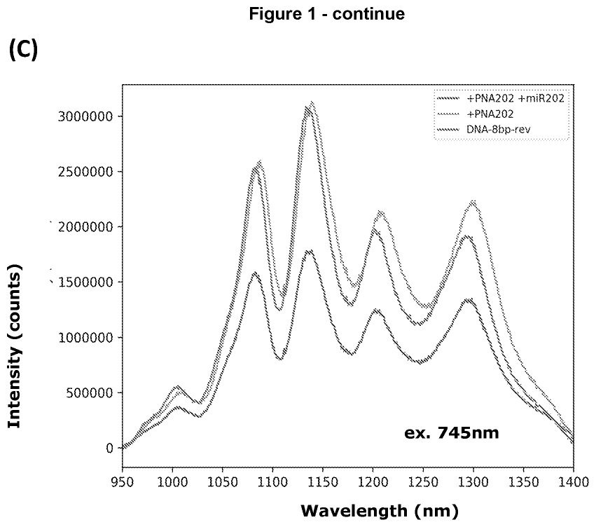

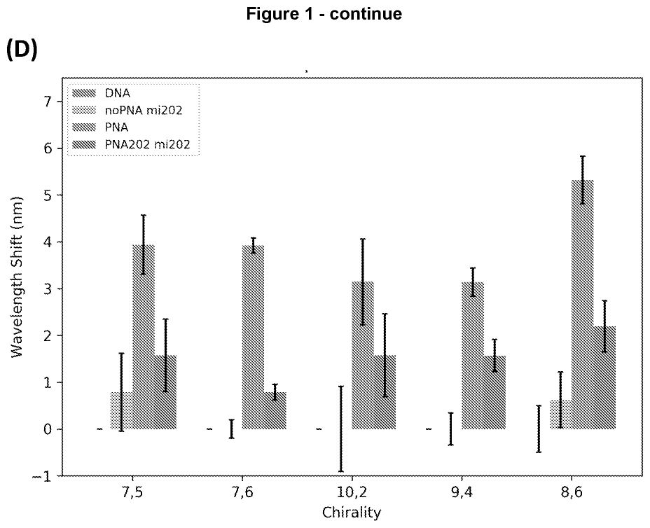

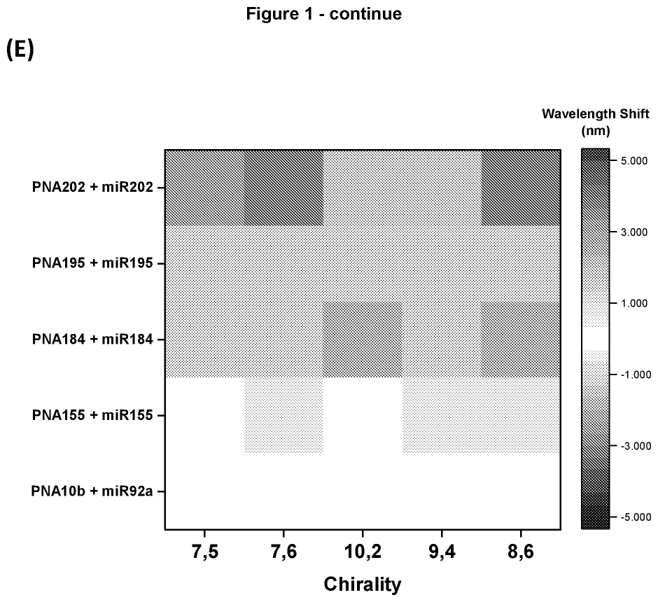

In order to maximize the interaction of PNA with the DNA that was adsorbed onto the nanotube surface, free DNA was removed from the suspension using ultrafiltration devices. PNA-DNA-SWCNTs were constructed by mixing PNA solution together with the DNA-SWCNT suspension. A schematic of the assembly process for the PNA-DNA-SWCNTs is shown in FIG. 1 (A).

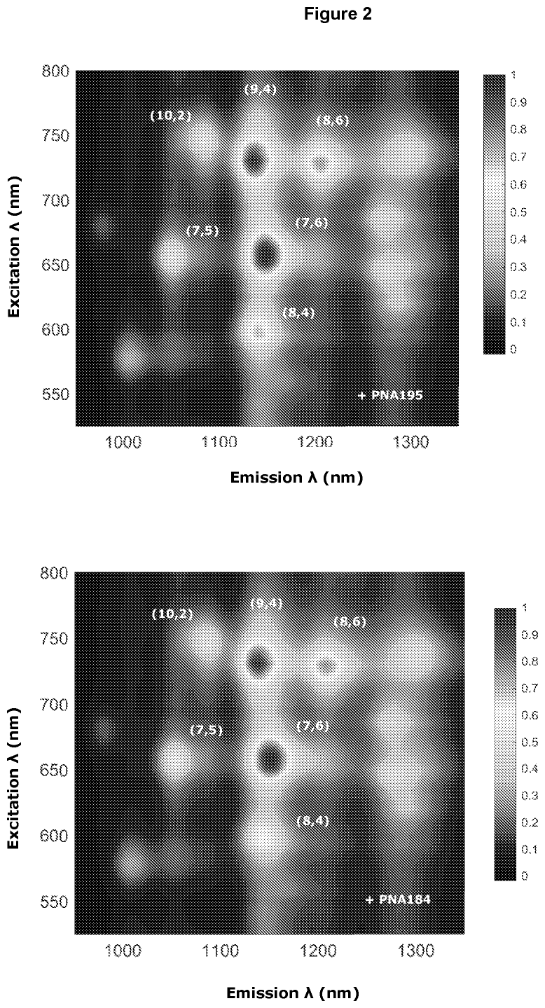

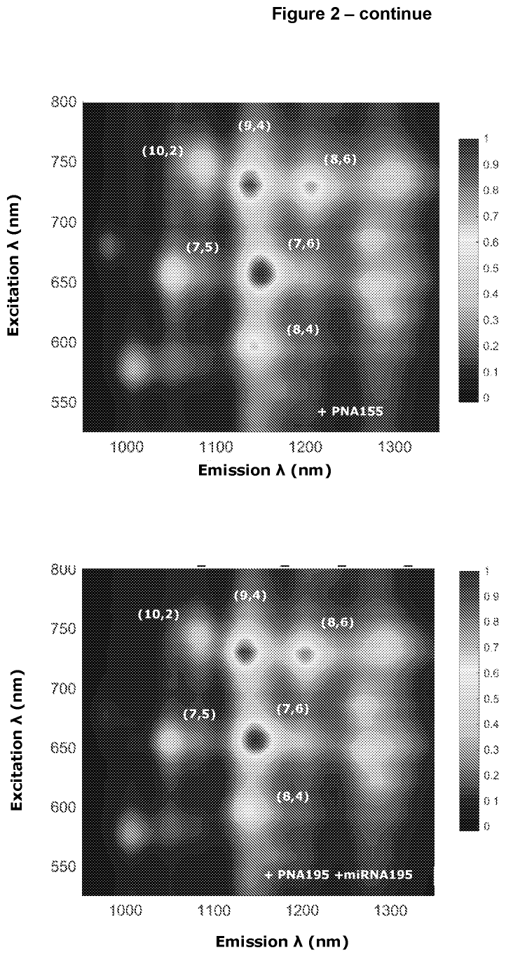

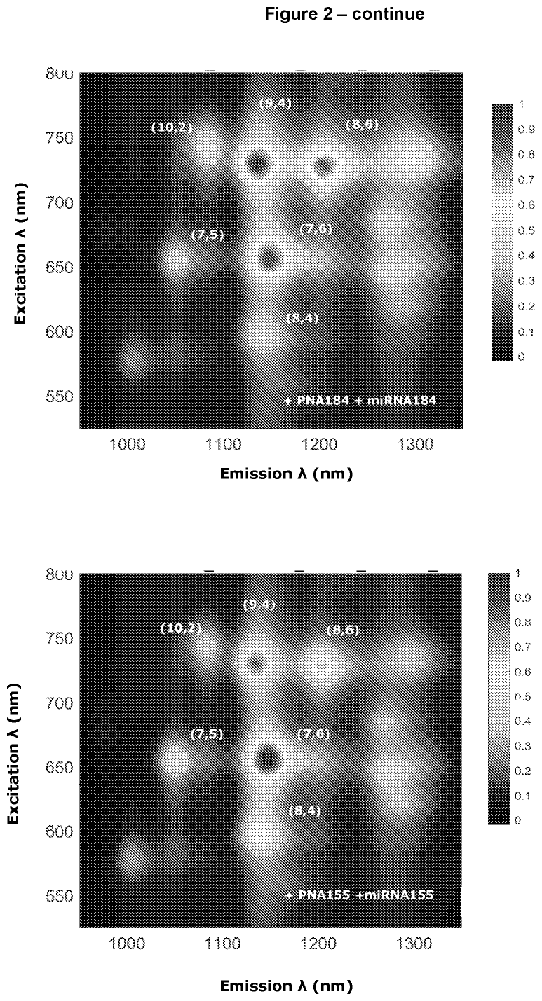

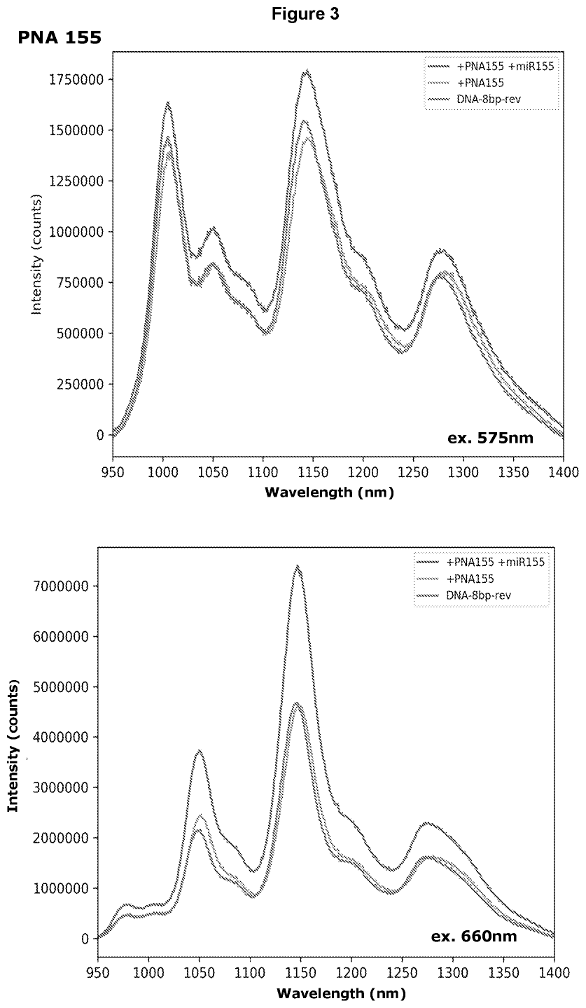

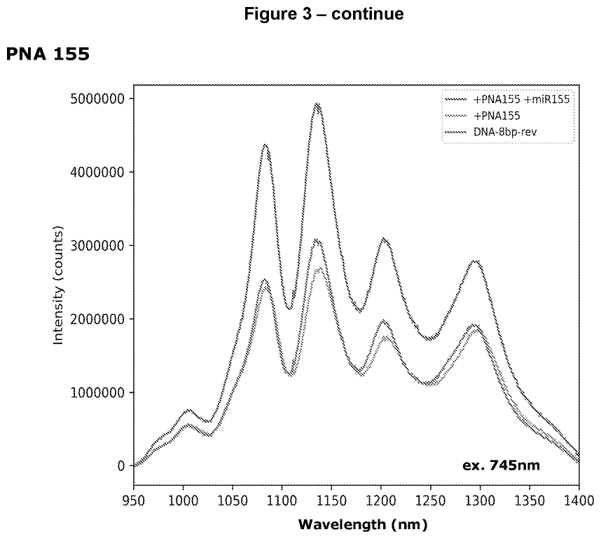

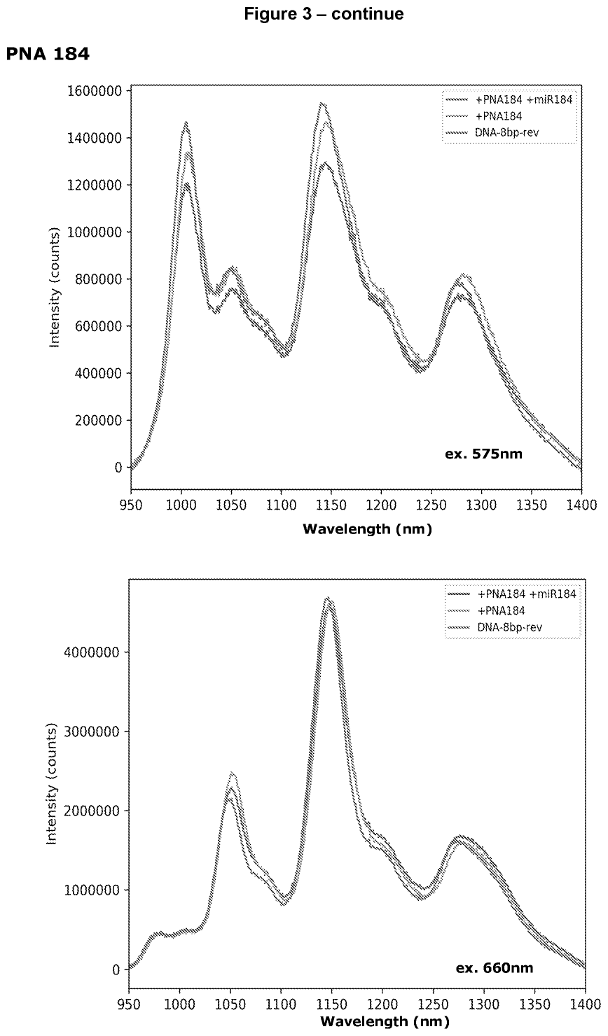

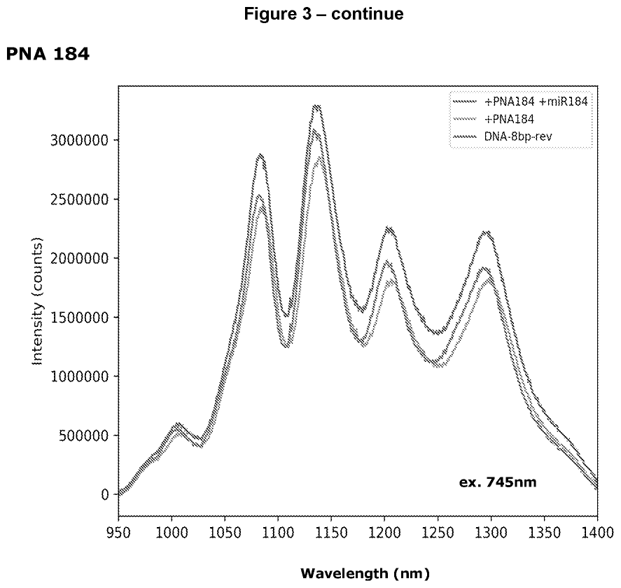

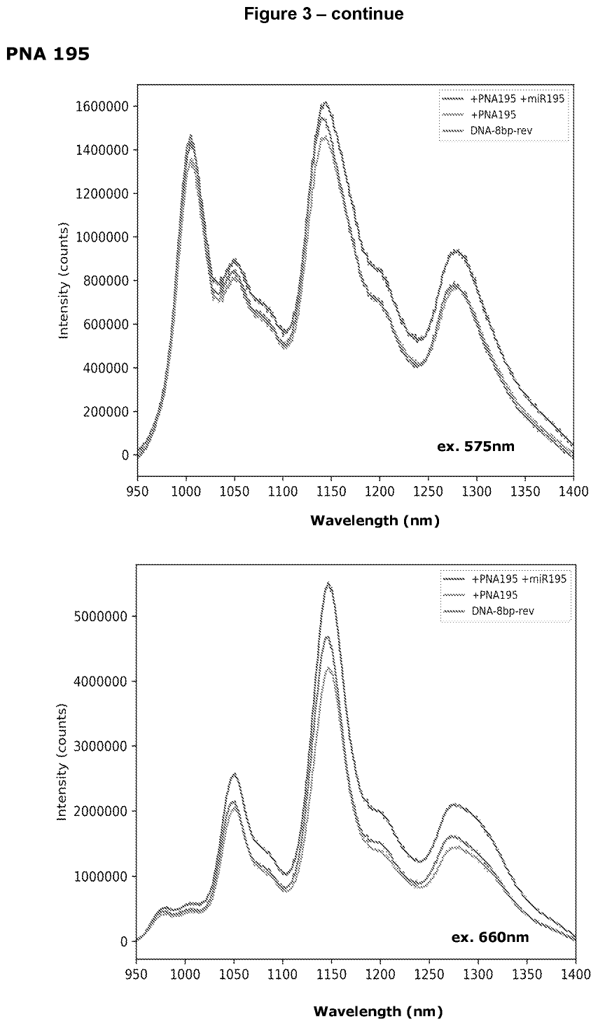

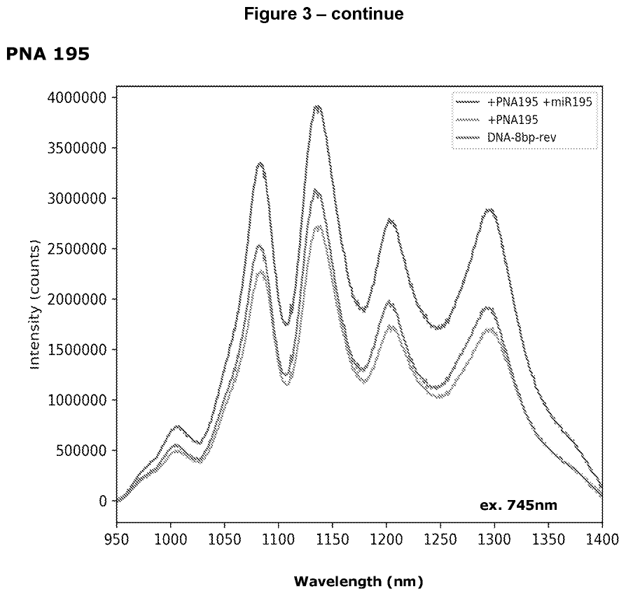

The optical response of DNA_{8bp-rev}-SWCNTs following the addition of PNA or PNA with miRNA was examined using fluorescence spectroscopy. All samples were incubated for a period of 1-2 h following addition of miRNA and PNA prior to acquiring any spectra. As shown in the photoluminescence excitation/emission (PLE) maps (FIG. 1 (B), FIG. 2), distinct differences were observed following the addition of PNA and following the subsequent addition of complementary miRNA for all PNA-DNA-SWCNTs. Line plots were extracted from the PLE maps to more easily compare the fluorescence changes on addition of PNA or miRNA (FIG. 1 (C), FIG. 3).

In general, the addition of PNA (final concentration: PNA=9 μM) resulted in a red-shift in the wavelength position corresponding to the peak fluorescence of the predominate chiralities. Despite this, the extent of the red-shift was sequence dependent; PNA₂₀₂ resulted in the largest shift (5.3±0.5 nm) and PNA195 the smallest shift (0.6±0.5 nm) both for the (8,6) chirality (all other wavelength shifts are listed in Table 1).

TABLE 1
Wavelength shift following the addition
of PNA sequences to DNA_{8 bp-rev}-SWCNTs

| PNA | Wavelength shift (nm) | | | | |
|---|---|---|---|---|---|
| sequence | (7, 5) | (7, 6) | (10, 2) | (9, 4) | (8, 6) |
| PNA_{10b} | 1.6 ± 0.8 | 0.8 ± 0.2 | 1.6 ± 0.9 | 1.6 ± 0.4 | 1.4 ± 0.5 |
| PNA₁₅₅ | 2.4 ± 0.8 | 1.6 ± 0.2 | 1.6 ± 0.9 | 1.6 ± 0.4 | 2.2 ± 0.5 |
| PNA₁₈₄ | 2.4 ± 0.8 | 2.4 ± 0.2 | 1.6 ± 0.9 | 1.6 ± 0.3 | 3.0 ± 0.5 |
| PNA₁₉₅ | 0.0 ± 0.8 | 0.0 ± 0.2 | 0.0 ± 0.8 | 0.0 ± 0.3 | 0.6 ± 0.6 |
| PNA₂₀₂ | 3.9 ± 0.6 | 3.9 ± 0.2 | 3.1 ± 0.9 | 3.1 ± 0.3 | 5.3 ± 0.5 |

The addition of PNA₂₀₂ resulted in the largest red-shift of any of the PNA sequences for all chiralities. Increased water accessibility or increased anionic charge is known to result in red-shifting of nanotube emission wavelengths, while decreases in local anionic charge density or water density at the nanotube surface can cause blue-shifting. As PNA is uncharged, this red-shifting may be attributed to an increase in the accessible surface area (decrease of surface coverage), which results in increased water accessibility to the nanotube surface.

It was hypothesized that this increase in surface area is a result of the dissociation of the partial duplex formed from the nanotube surface. The larger increase relative to the other PNA sequences may result from the shorter sequence length of PNA₂₀₂. For PNA₂₀₂-DNA_{8bp-rev}-SWCNTs, subsequent addition of complementary miRNA (miR202, final concentration: 1 μM) resulted in blue-shifting of the fluorescence back towards the original positions of the DNA_{8bp-rev}-SWCNTs. Importantly, the addition of miR202 in the absence of PNA₂₀₂ did not result in any significant shifting of the wavelength position for any of the chirality peaks measured (FIG. 1 (D)) indicating that the response observed was due to the interaction of the PNA and complementary miRNA sequence. While it is possible that PNA preferentially binds with miRNA before fully hybridizing with the DNA-SWCNT or that post-hybridization the PNA complex detaches from the DNA_{8bp-rev}-SWCNT, the strong stability of PNA-DNA complexes means this is unlikely even accounting for the increased stability of the PNA-RNA complex. Instead, the blue-shift of PNA₂₀₂ following miR202 addition may be due to increased surface coverage following hybridization, which can be associated to the PNA-miRNA duplex adsorbing onto the nanotube surface.

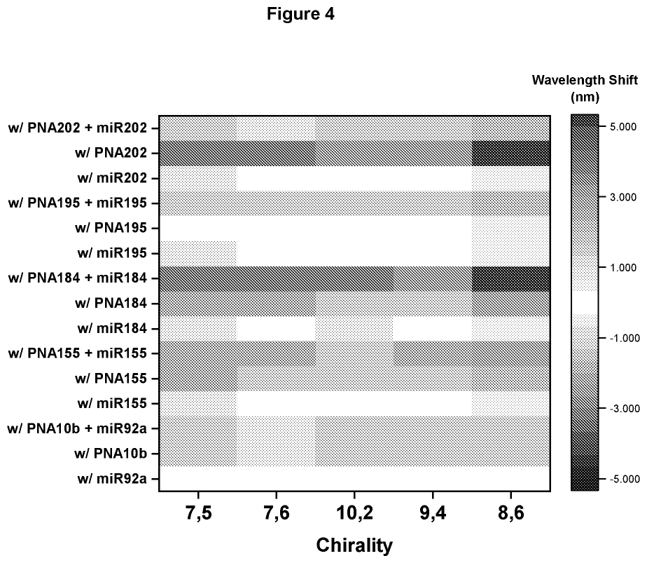

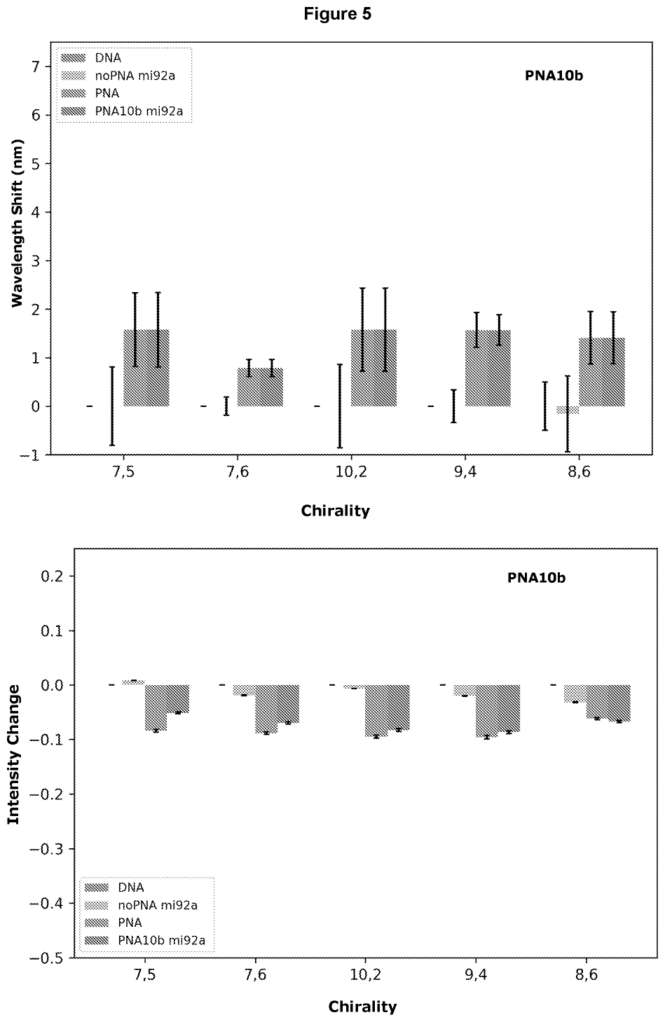

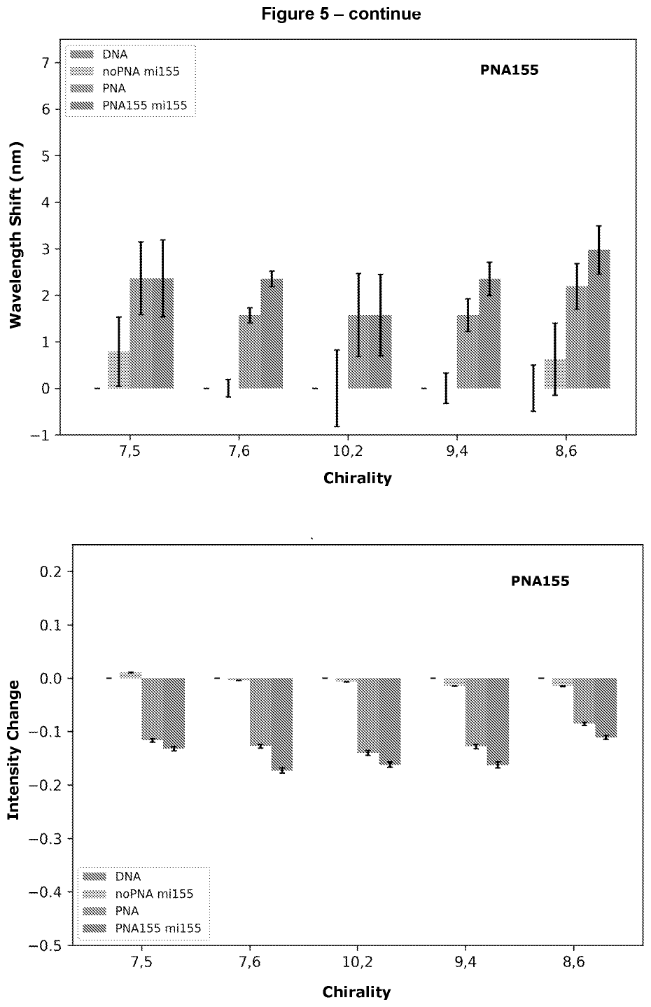

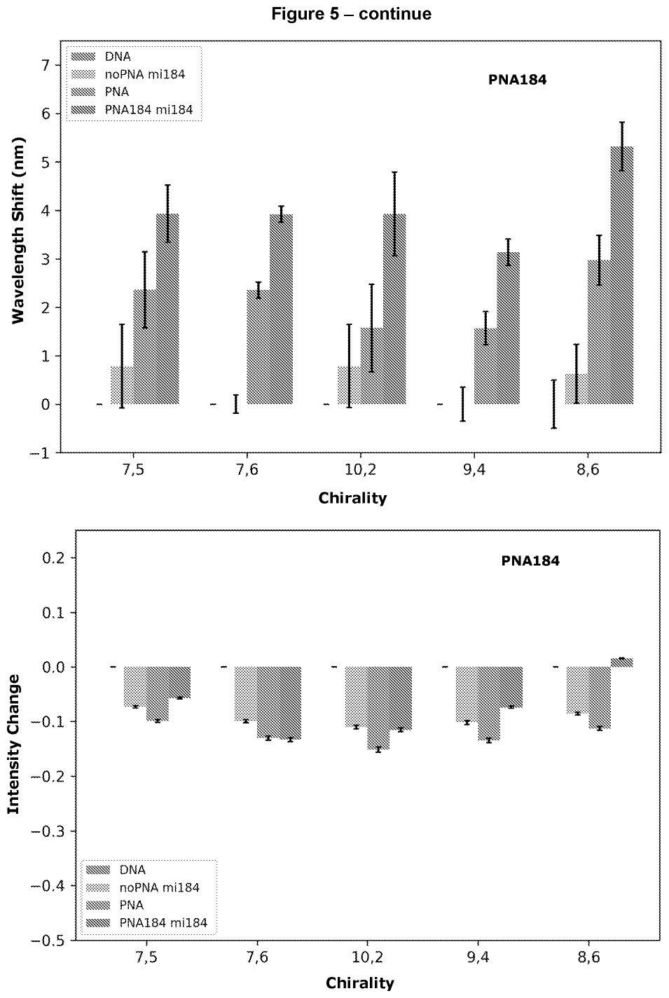

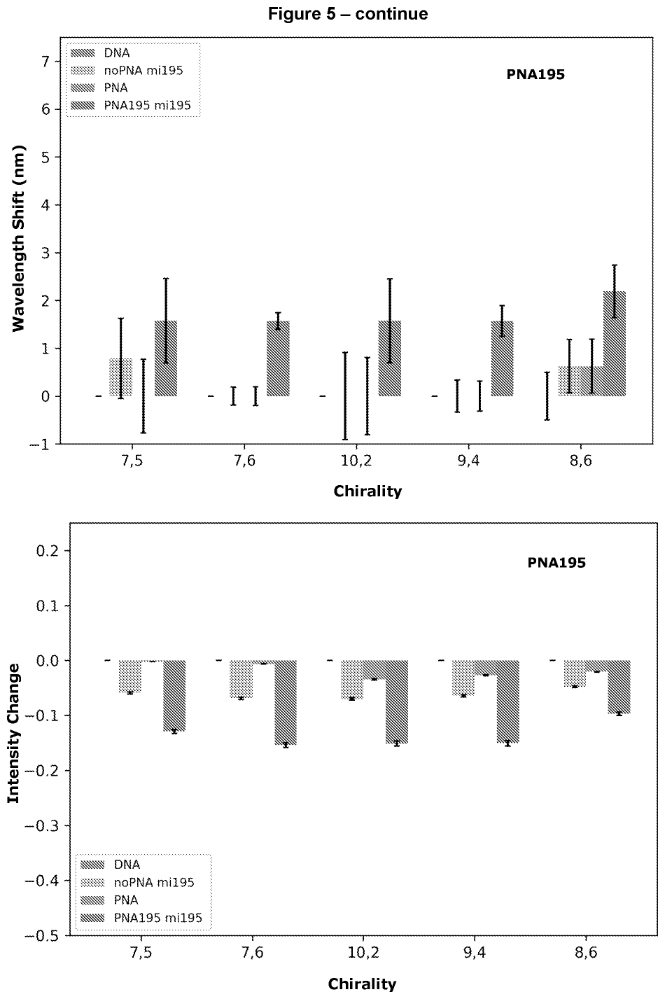

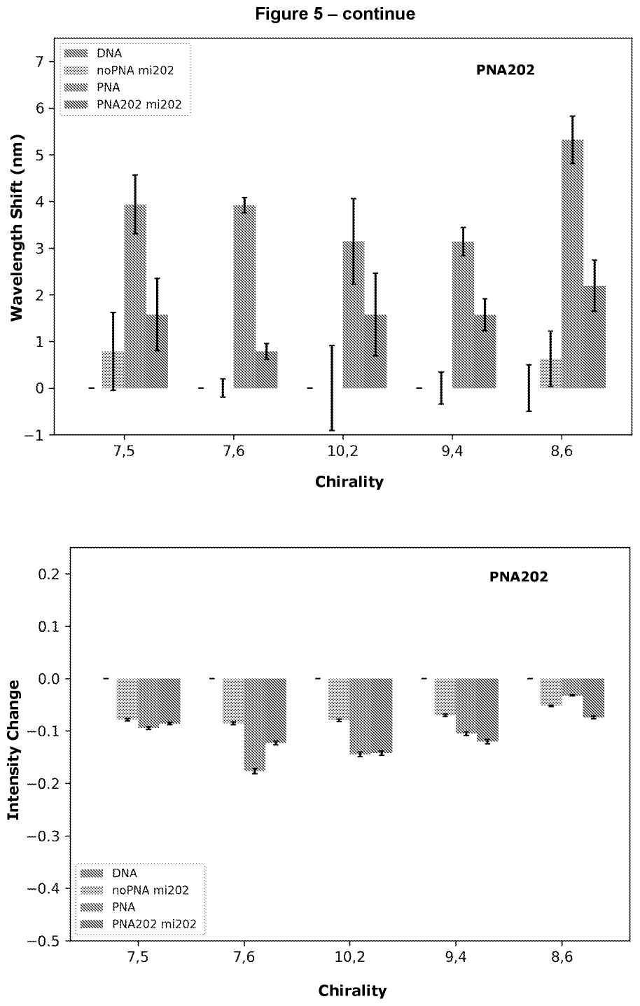

Conversely, for PNA₁₉₅, PNA₁₈₄, and PNA₁₅₅ the addition of complementary miRNA resulted in increased red-shifting (FIG. 1 (E), FIGS. 4, 5). This response continued to be statistically distinct from the optical response following miRNA addition in the absence of PNA for all sequences. Differences in the directionality of the shift may result from fundamentally different interaction mechanisms that result in unique structural changes depending on the PNA and miRNA combination. The enhanced red-shift may be the result of increases in the local anionic charge density either due to non-specific adsorption of the miRNA or from the phosphate backbone from the miRNA being brought into close proximity of the nanotube surface following hybridization. However, it is discounted the possibility that the red-shift is due to non-specific adsorption of the miRNA onto the nanotube due to a lack of significant shifting following miRNA addition in the absence of PNA (FIG. 4). Alternatively, the red-shift may result from increasing water accessibility due to partial desorption of the hybridized PNA-miRNA from the nanotube surface.

The addition of non-complementary miRNA (miR92a) to PNA_{10b} did not result in any significant wavelength shifts for any chirality measured, indicating that the interaction of the PNA and miRNA was sequence specific and providing further evidence that the red-shifts observed for the other PNA sequences were not a result on non-specific adsorption.

Following the initial observations using DNA_{8bp-rev}-SWCNTs, the inventors sought to examine the impact of the length of the hybrid portion, as well as the directionality of the PNA-DNA hybridization (parallel or anti-parallel), on the sensing capabilities of the PNA-DNA constructs.

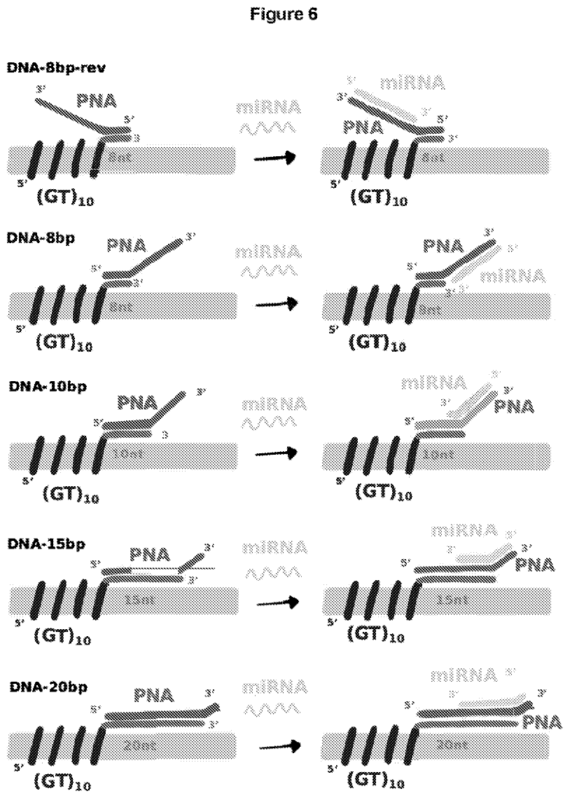

In contrast to DNA, PNA can bind in either a parallel or anti-parallel fashion as a result of its more flexible backbone. However, kinetic studies have shown that, in general, PNA demonstrates a preference towards the antiparallel binding orientation with lower affinity observed for duplexes formed with parallel orientation. In order to maximise the extent by which PNA preferentially binds with the target miRNA sequence over the DNA, the inventors predominately tested constructs that consistent of a DNA-PNA hybrid section with a parallel orientation for miRNA binding on recognition in an anti-parallel fashion, thus enabling significantly faster duplex formation for the PNA-miRNA duplex and ideally result in a more reactive complex. A schematic illustrating the different configurations of PNA-DNA-SWCNTs tested is provided in FIG. 6.

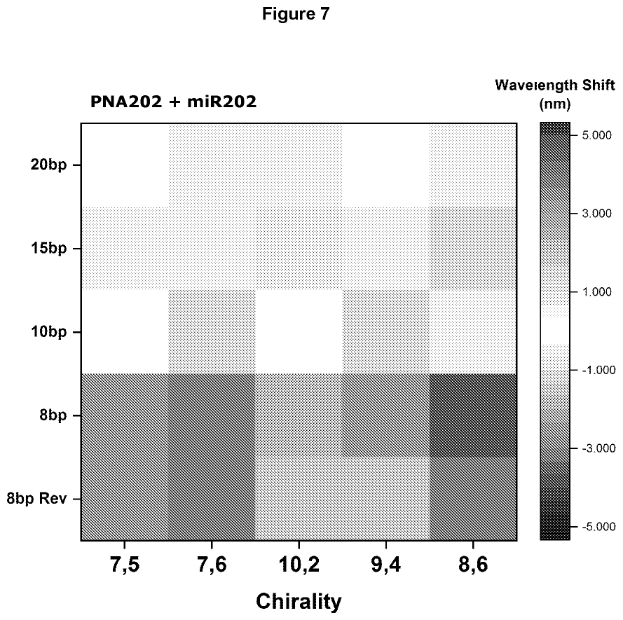

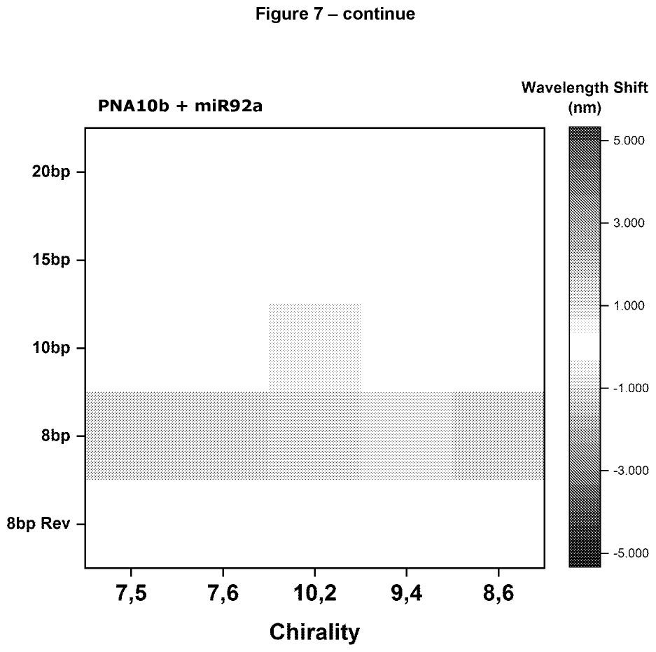

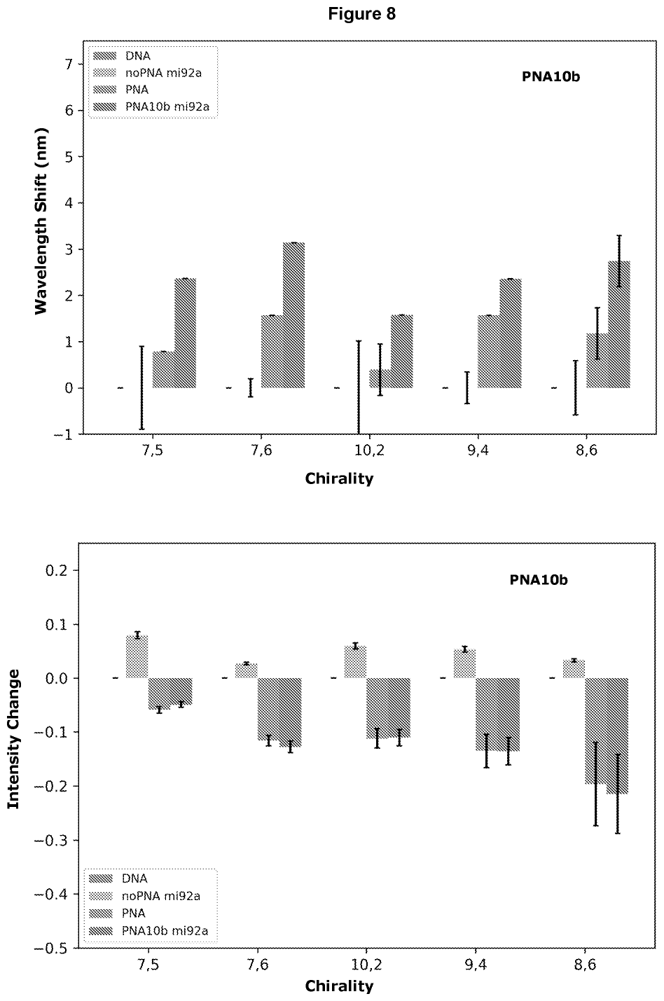

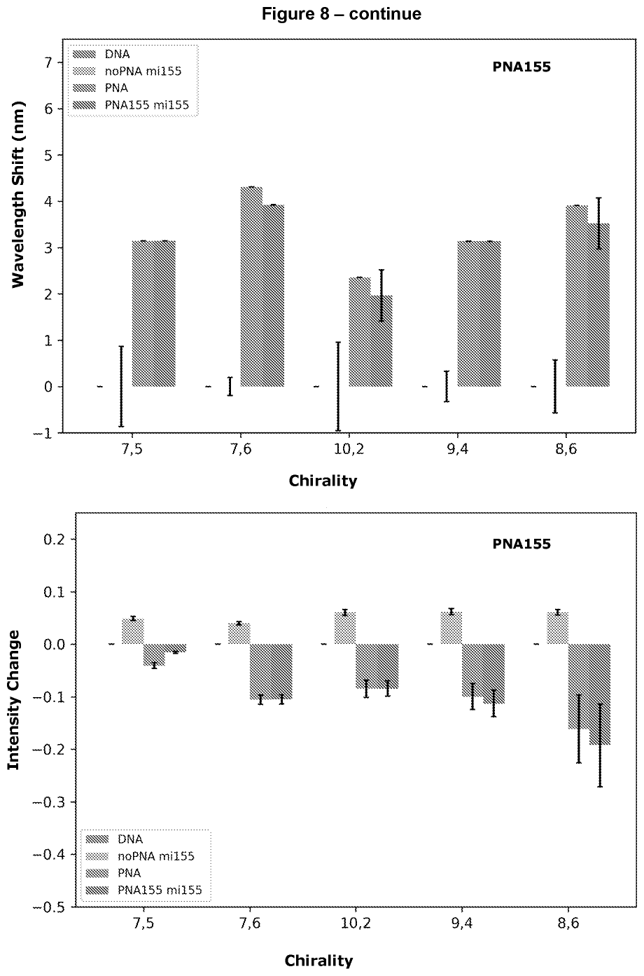

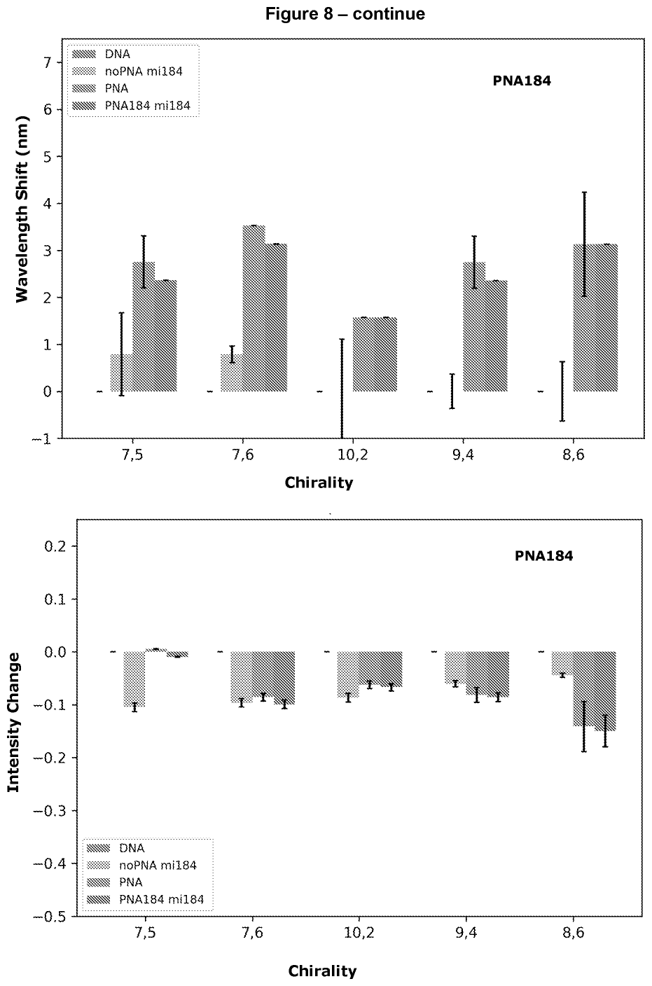

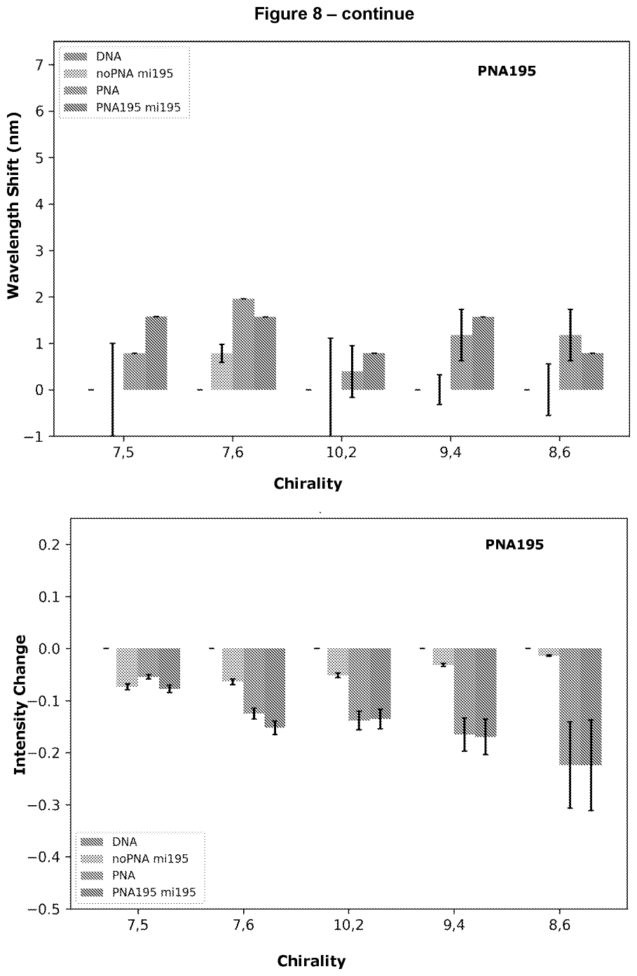

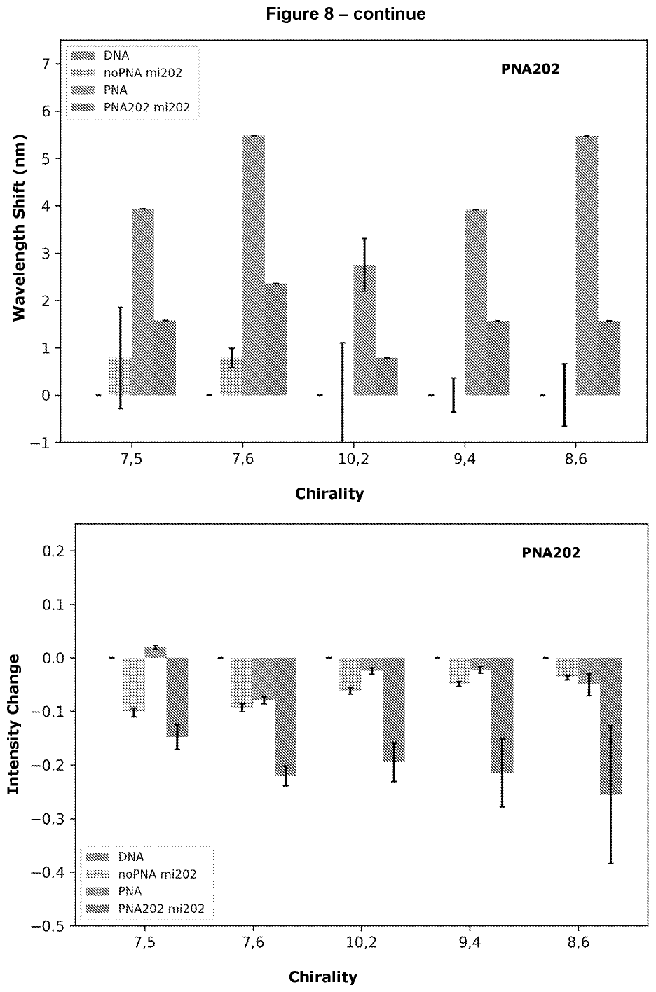

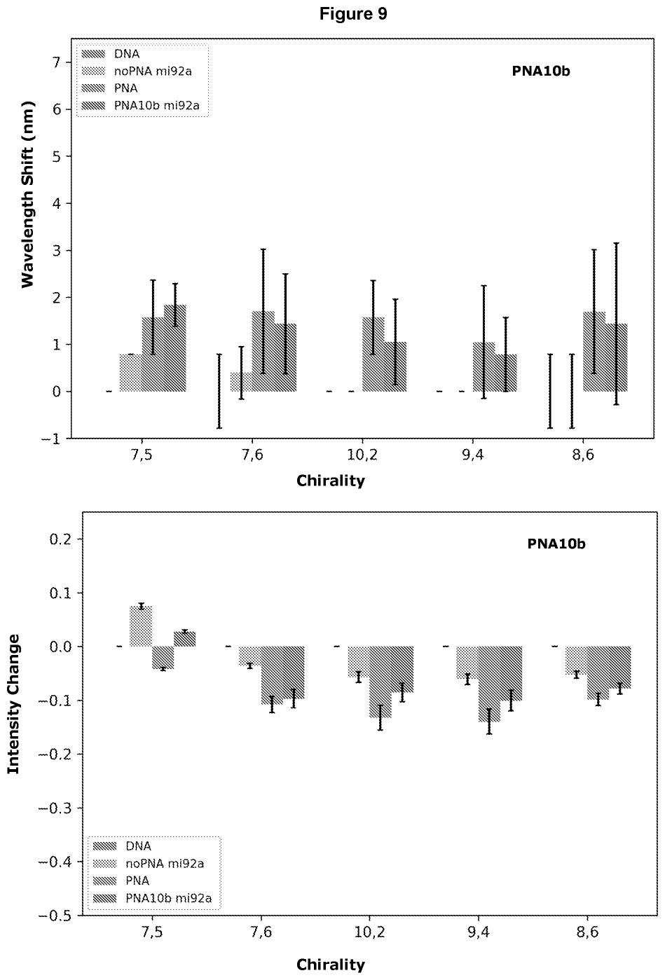

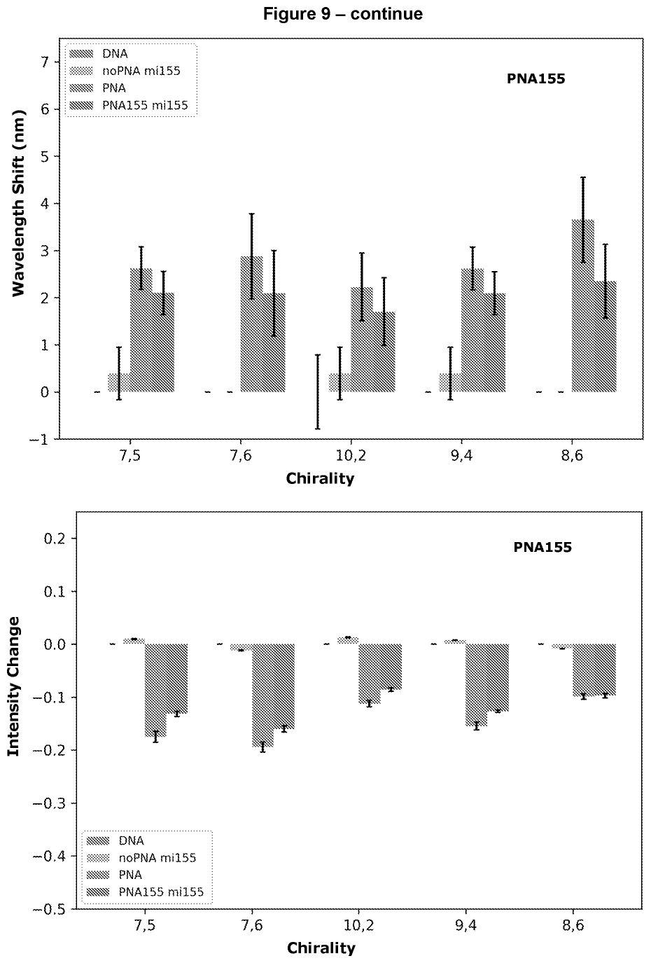

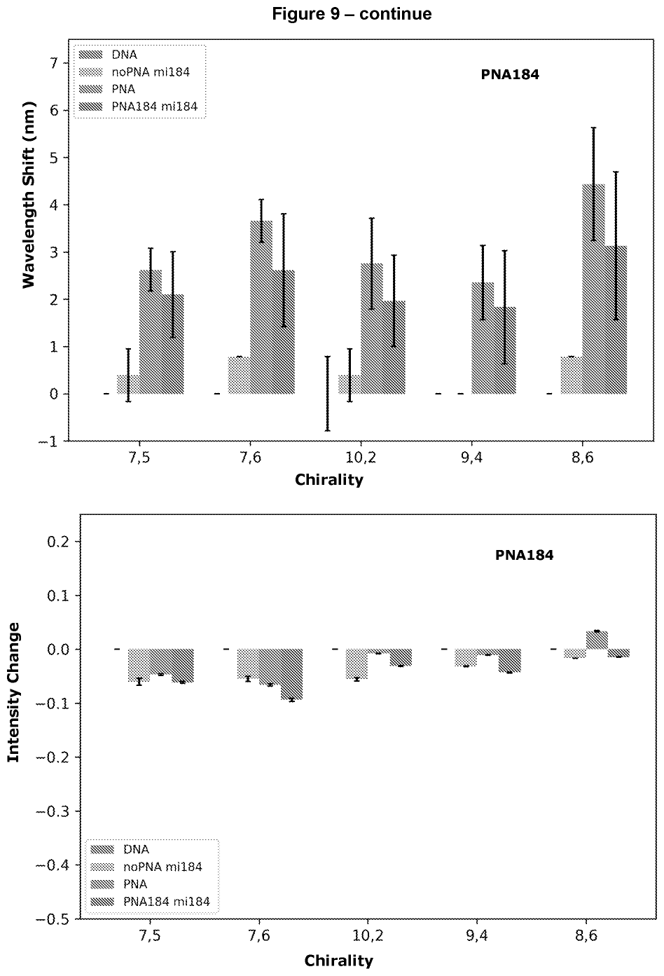

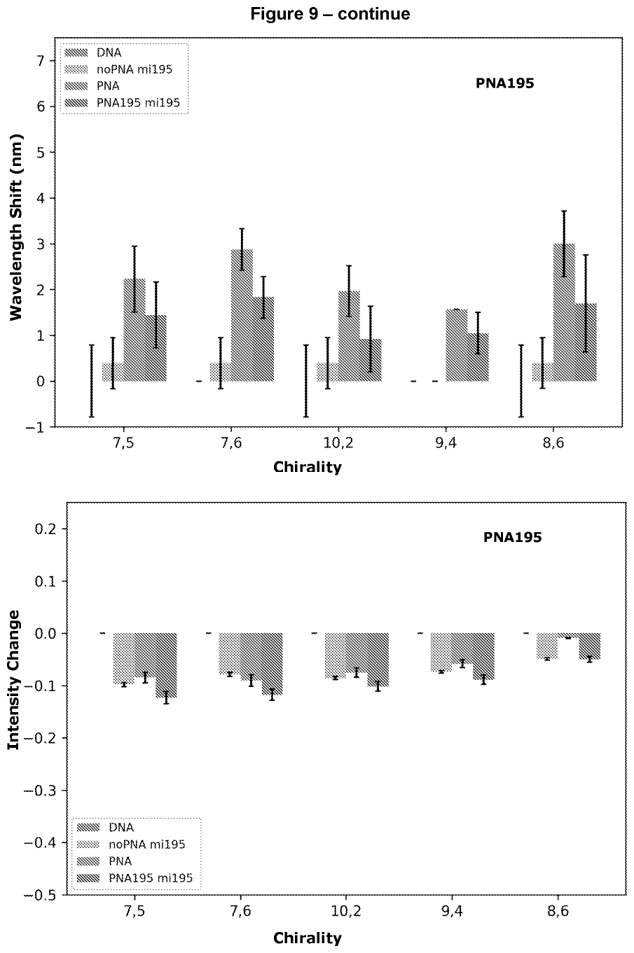

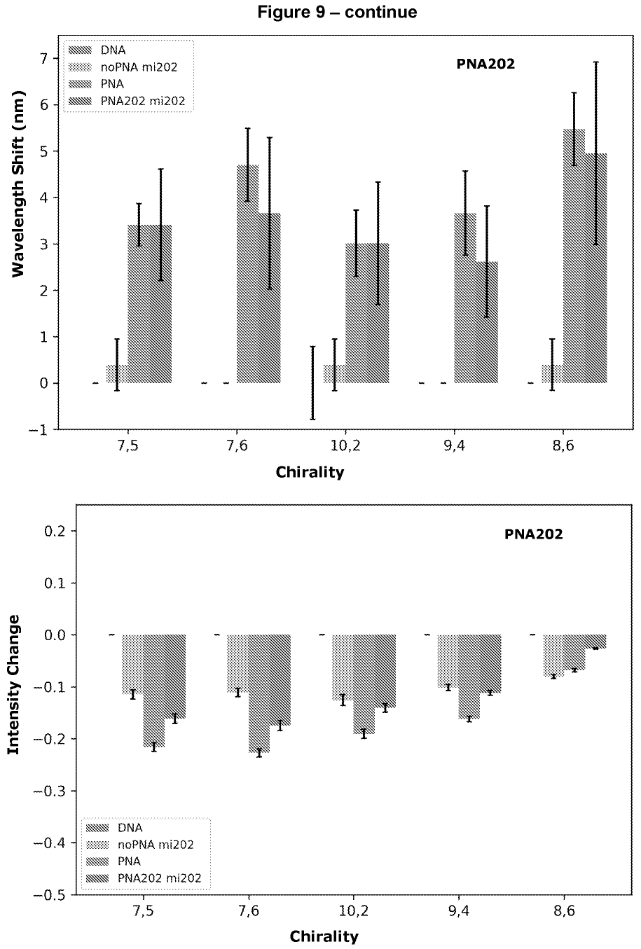

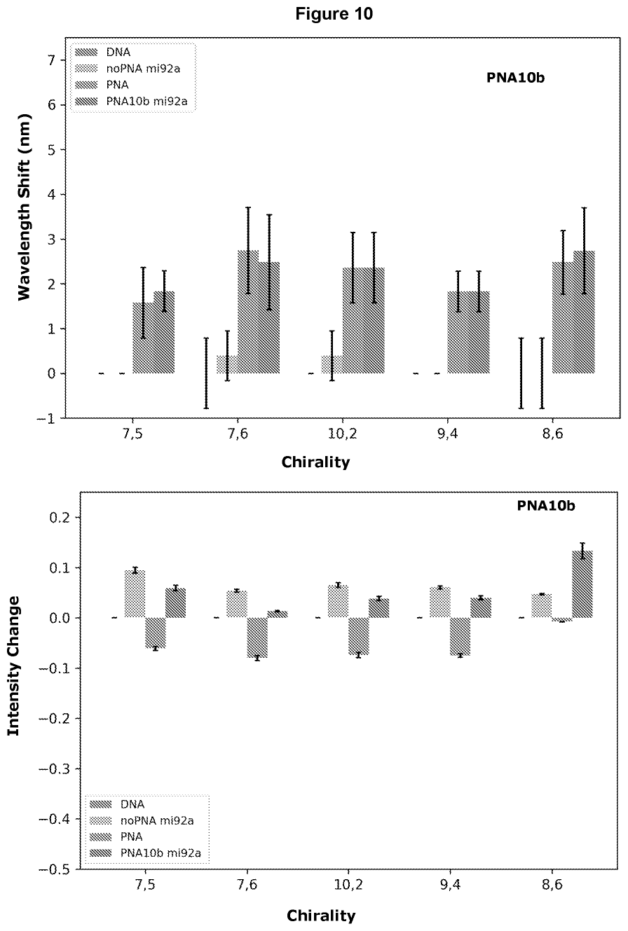

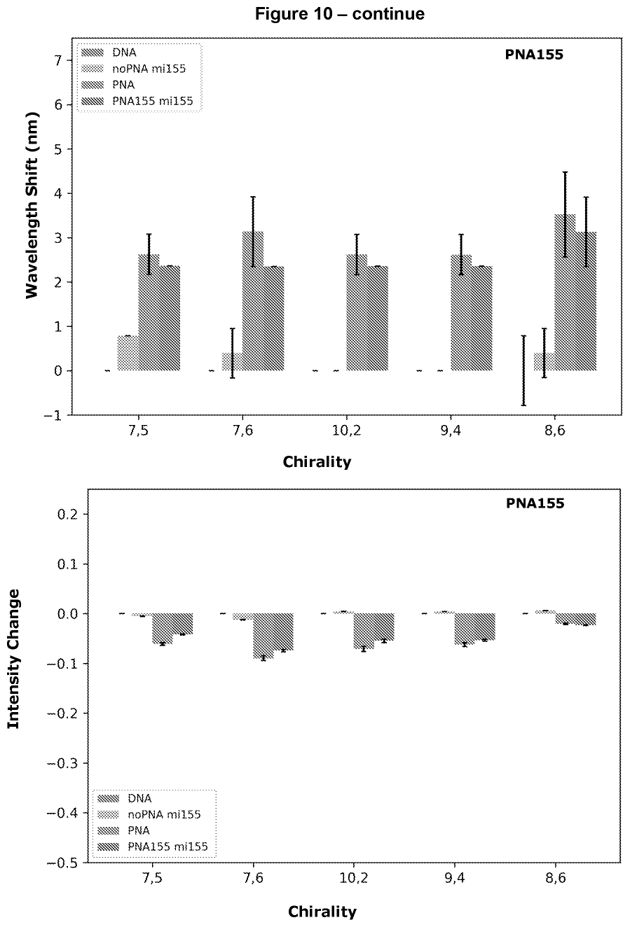

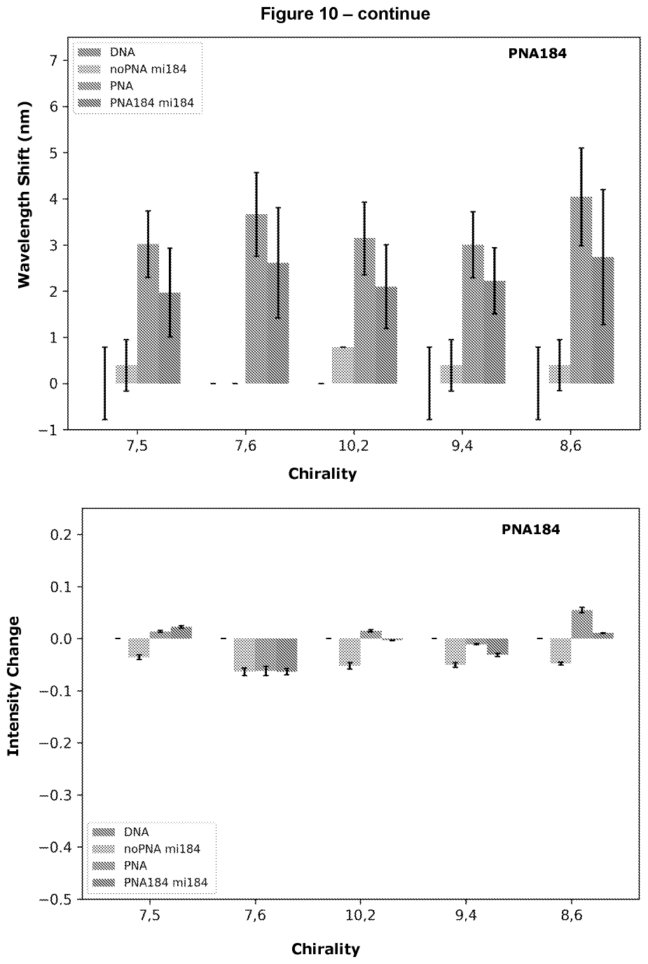

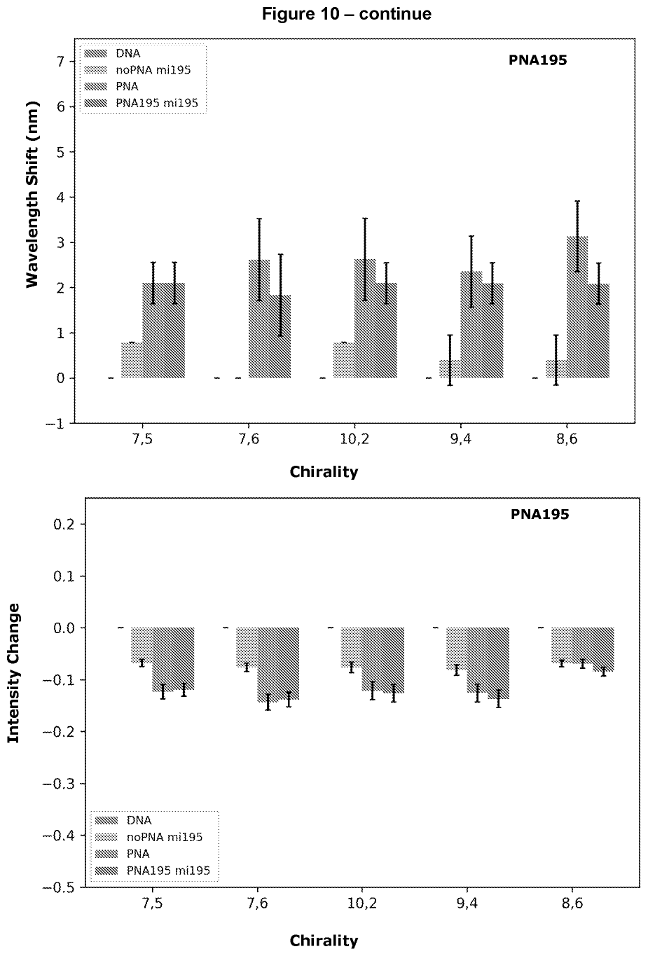

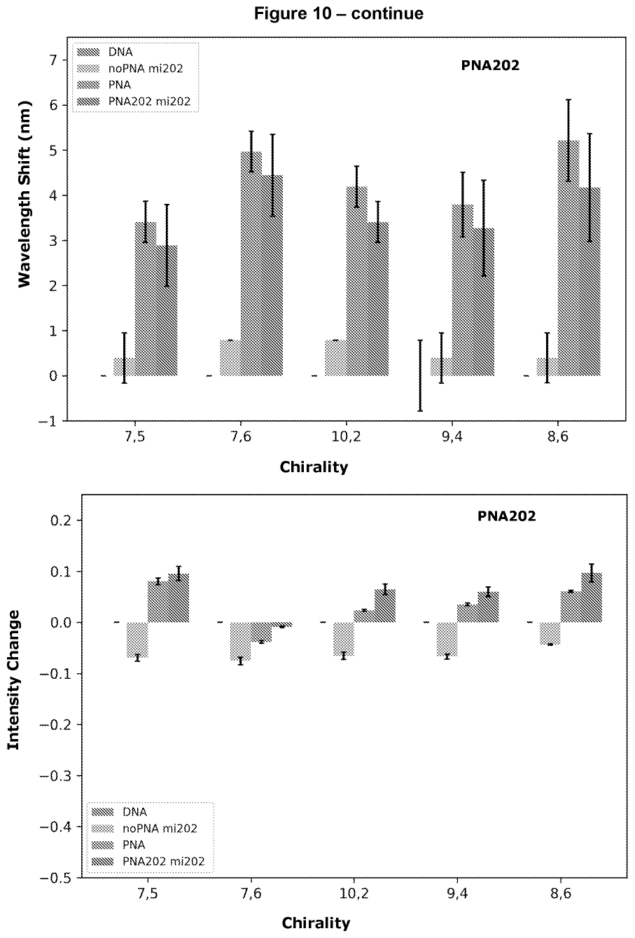

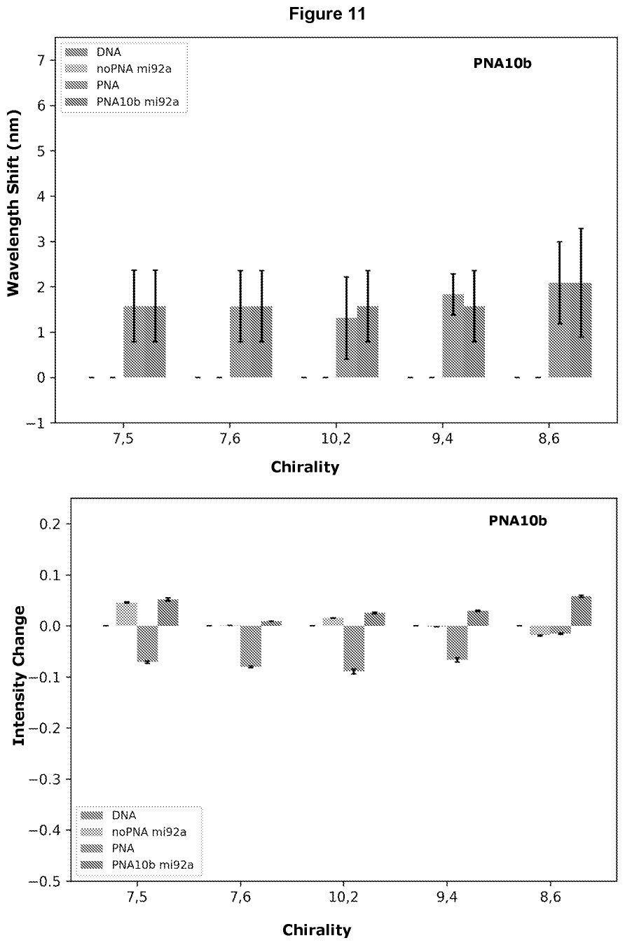

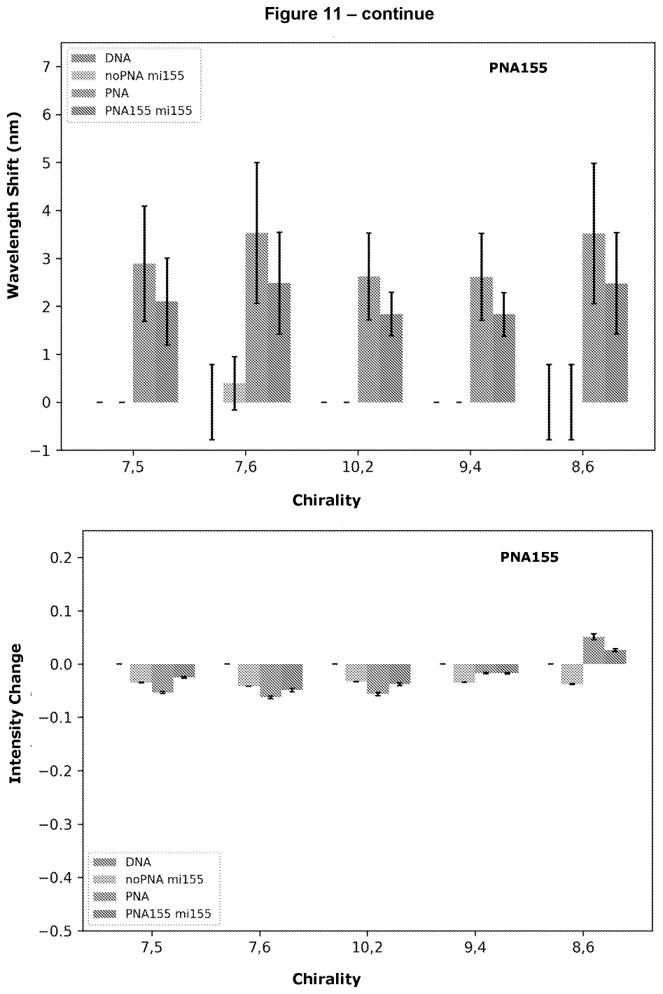

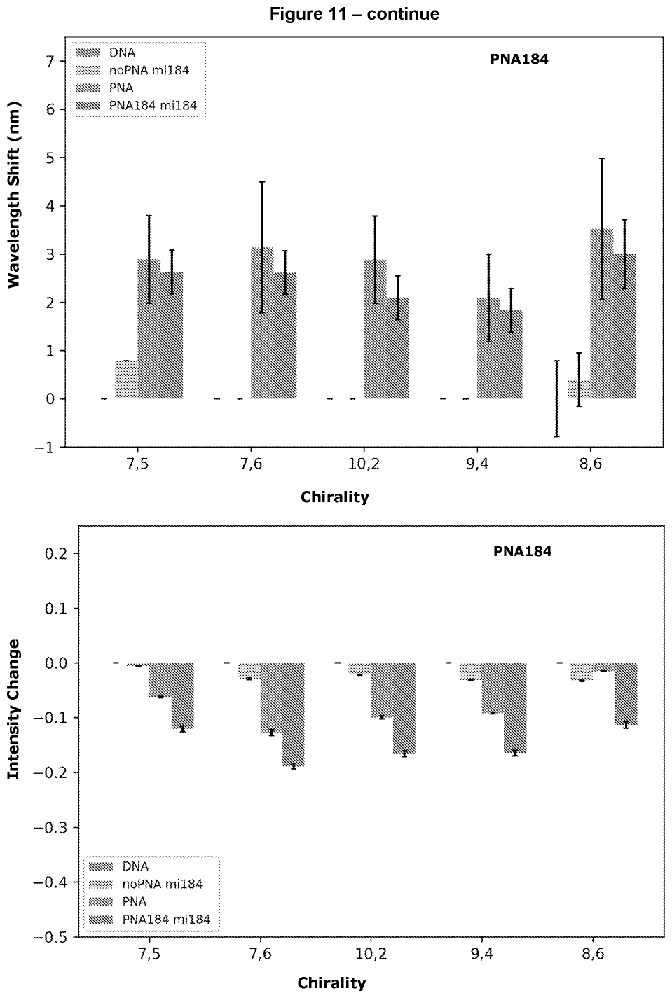

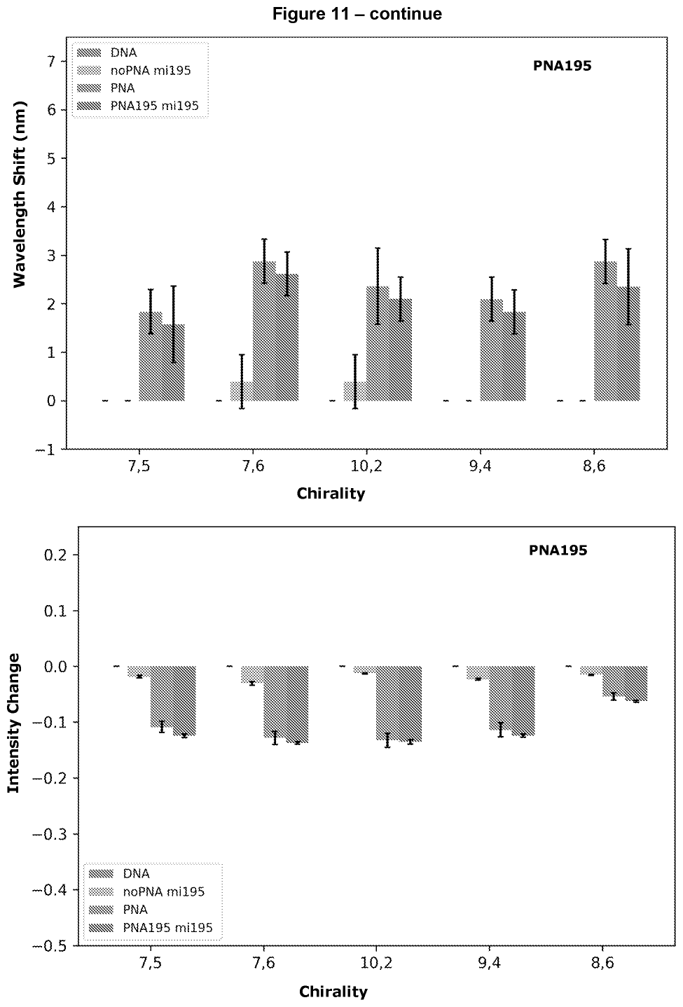

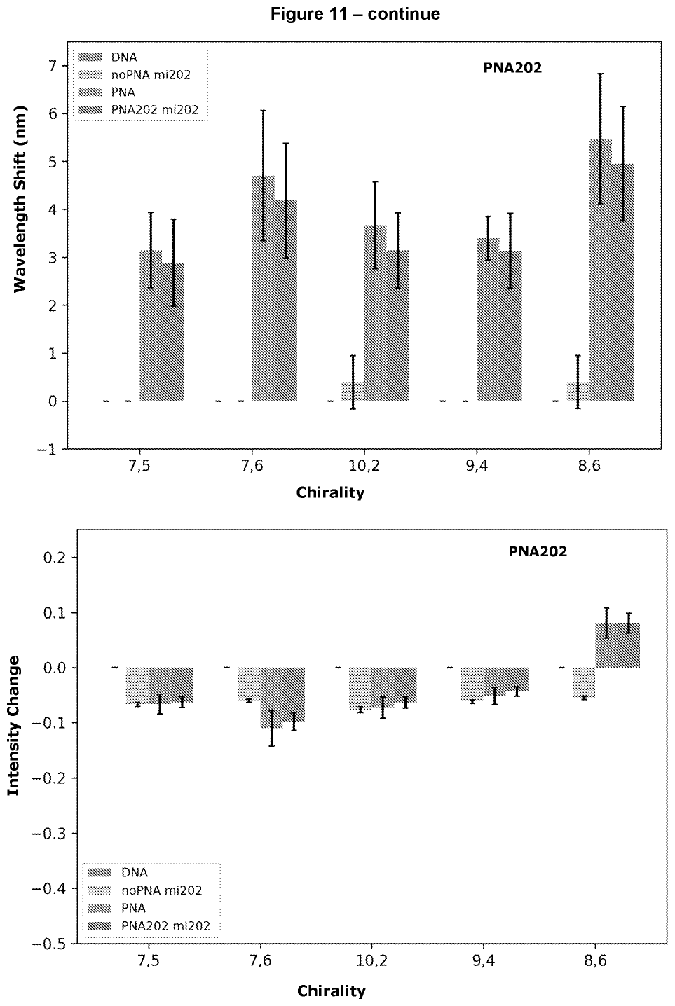

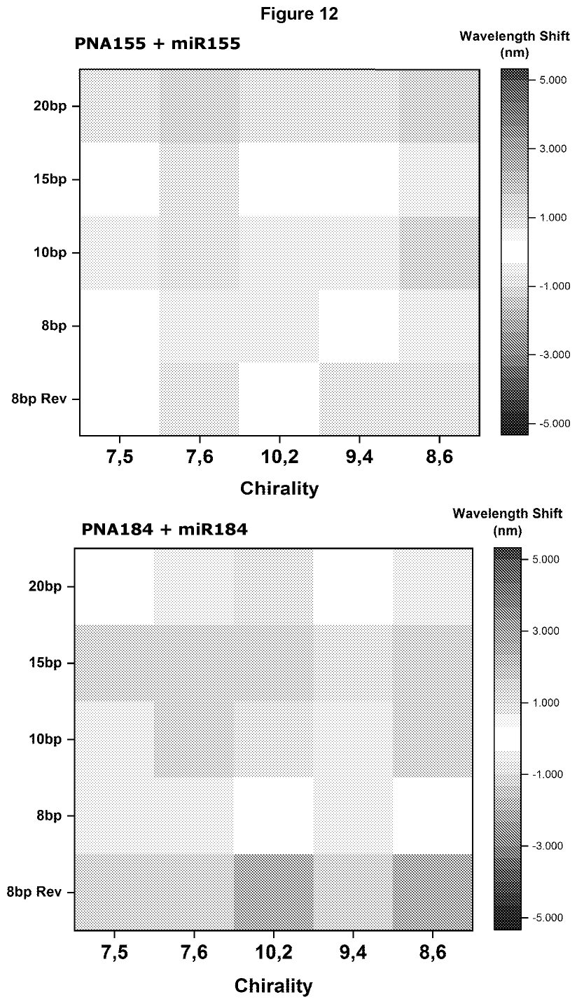

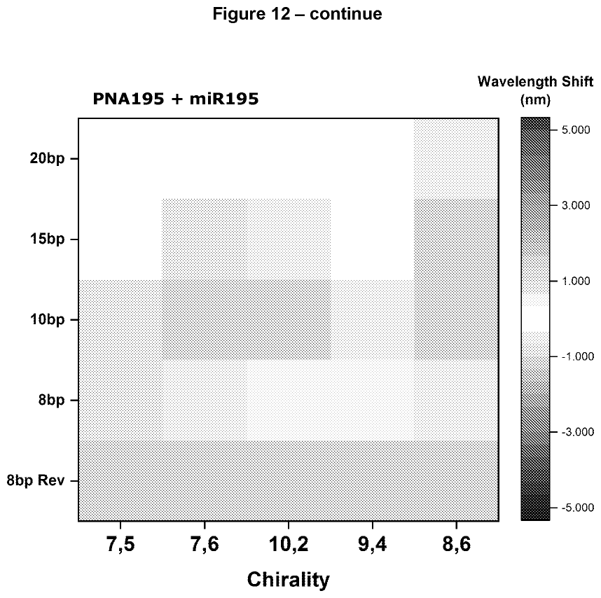

In line with expectations, the PNA202 complexes formed using DNA8bp-SWCNTs (parallel binding) had a larger blueshift in the wavelength peak positions of all chiralities following the addition of complementary miRNA compared to when using DNA_{8bp-rev}-SWCNTs (antiparallel binding) (FIG. 7, FIGS. 5, 8). Moreover, it was observed a notable decrease in the wavelength shifting response for PNA₂₀₂-DNA-SWCNTs as the length of the hybrid section was increased. Interestingly, whereas PNA₁₅₅, PNA₁₈₄, and PNA₁₉₅ exhibited a red-shift in response to complementary miRNA for complexes using the DNA8bp-rev sequence, complexes formed using the parallel binding sequence, DNA8 bp, exhibited a blue-shifting response for certain chiralities depending on the PNA sequence. Furthermore, as the length of the hybrid section was increased, all PNA-DNA-SWCNTs began to exhibit a blue-shift in the wavelength position of all chiralities following the addition of complementary miRNA (FIGS. 5, 8, 9, 10, 11, 12). On the contrary, PNA_{10b}-DNA-SWCNTs, which are used as a negative control with non-complementary miRNA, showed no significant shifting following miRNA addition for any PNA-DNA complexes with a hybrid section length of 10 or more bases (FIG. 7).

However, a redshift in the peak positions of all chiralities was observed following the addition of non-complementary miRNA (miR92a) for the PNA_{10b}-DNA_{8bp}-SWCNTs. Although the exact reason for this observed wavelength shift remains an ongoing area of study, the possibility that it is due to non-specific adsorption of the miRNA onto the nanotube surface was rule out, as no significant changes were observed following miRNA addition in the absence of PNA (FIG. 8).

In order to examine whether a fluorescence wavelength shift response could be engineered for the PNA-DNA-SWCNTs with longer hybrid sections, the inventors increased the concentration of miRNA relative to the PNA. It was hypothesized that in order to see significant fluorescence responses a majority of the PNA-DNA-SWCNTs would need to react with miRNA. Based on this assumption, the response to miRNA could be recovered (or enhanced) by increasing the relative concentration of the miRNA to at least 50% of the PNA concentration in solution.

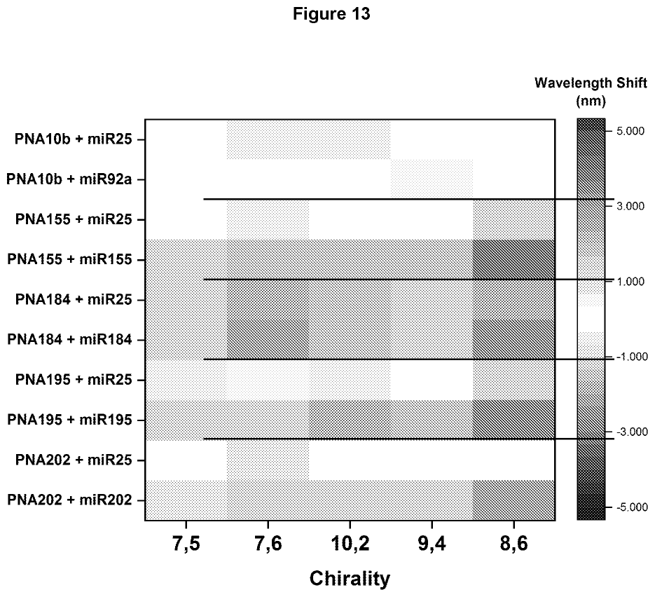

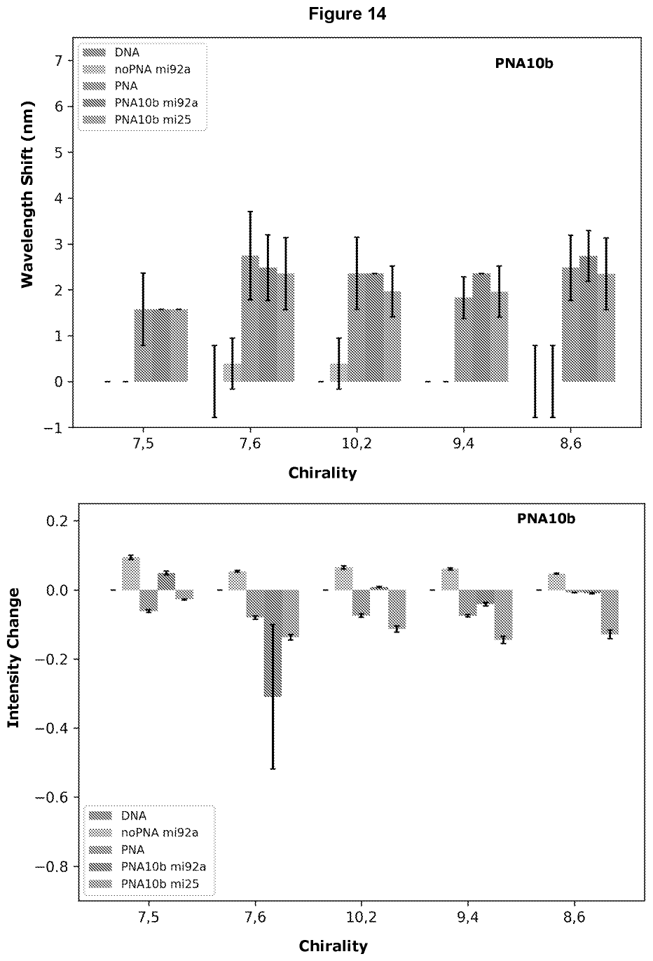

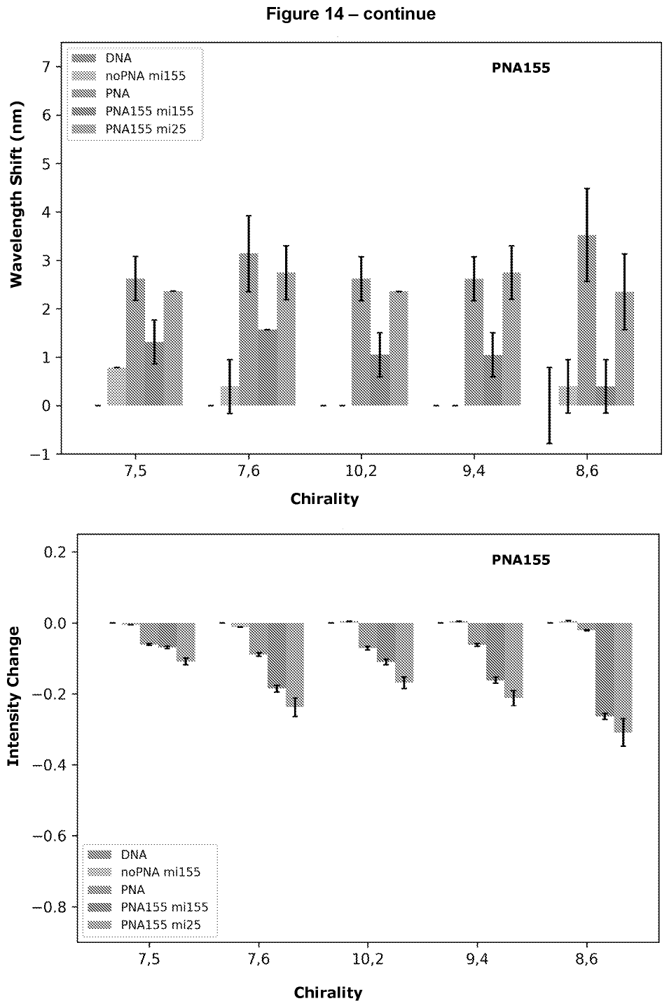

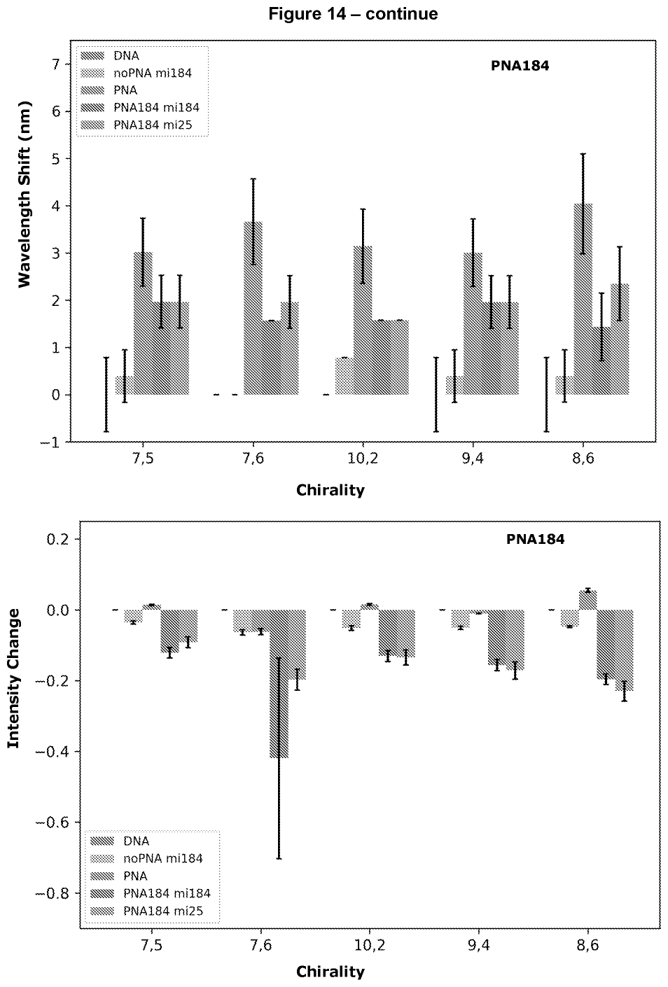

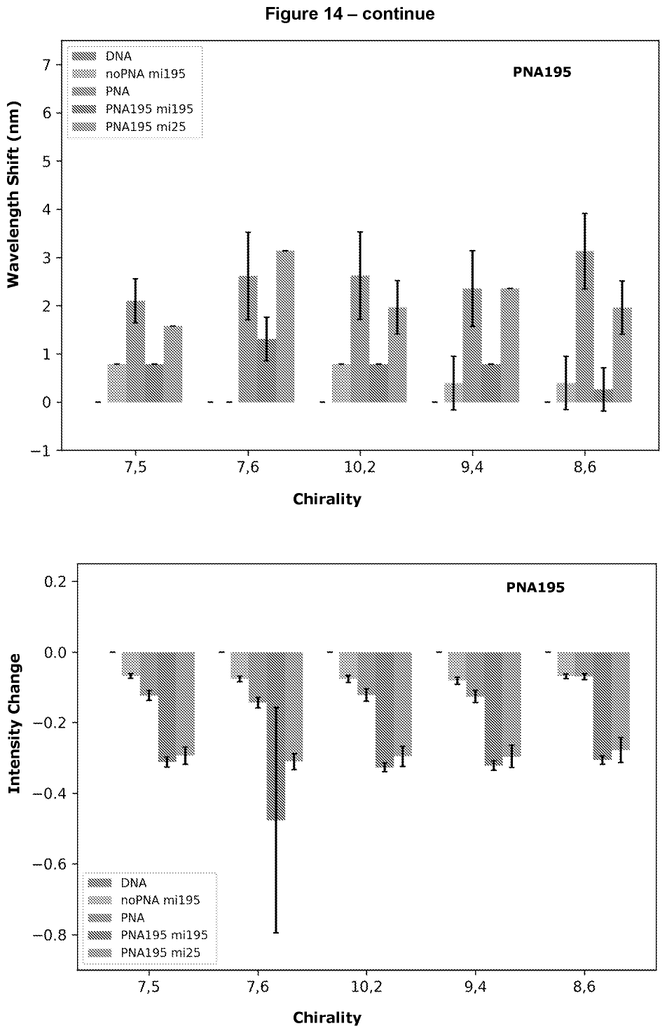

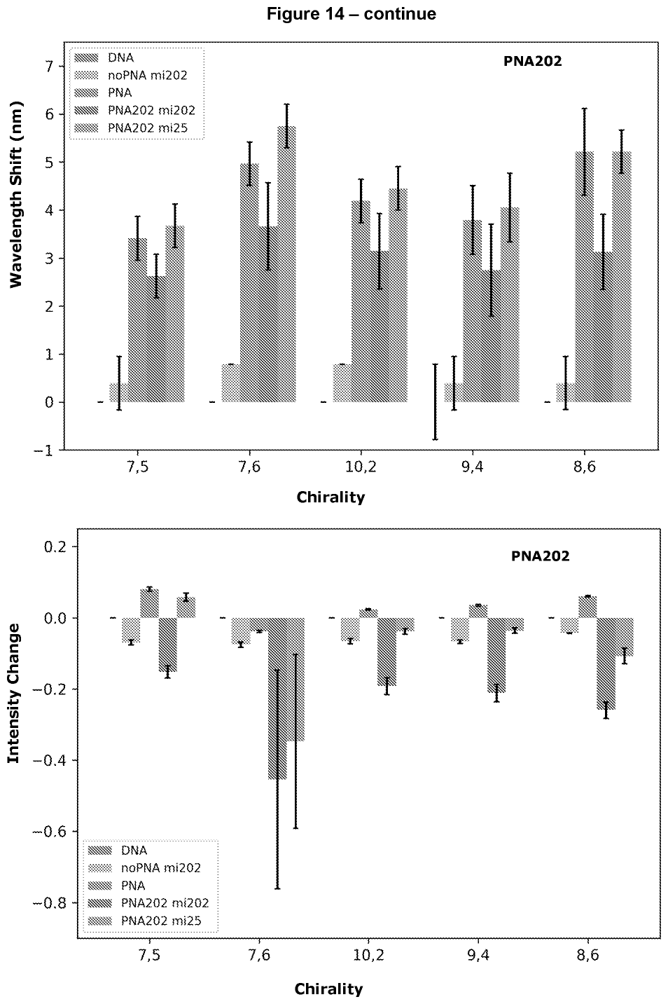

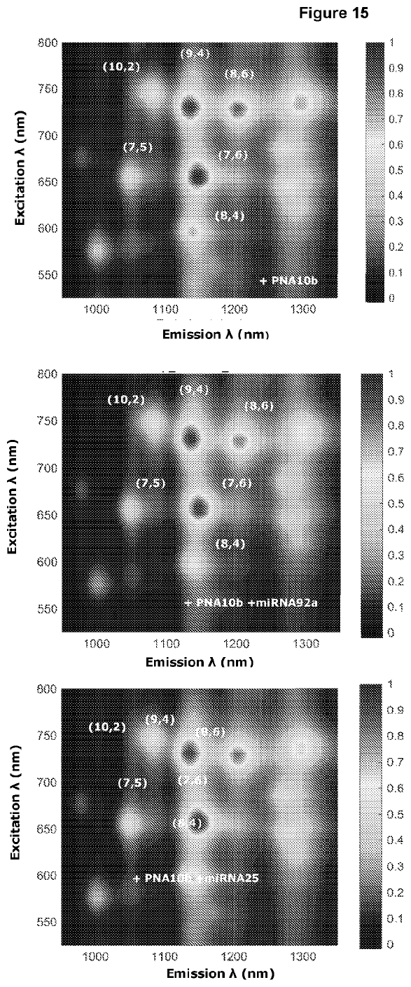

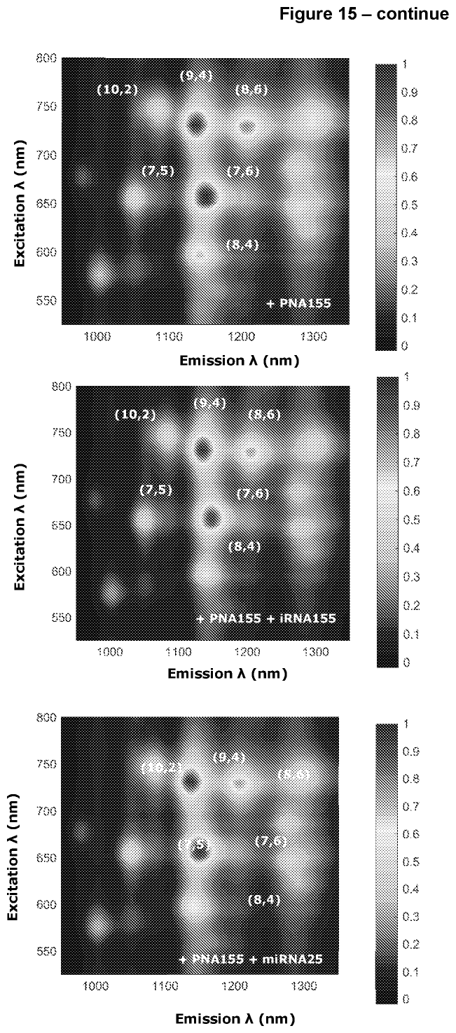

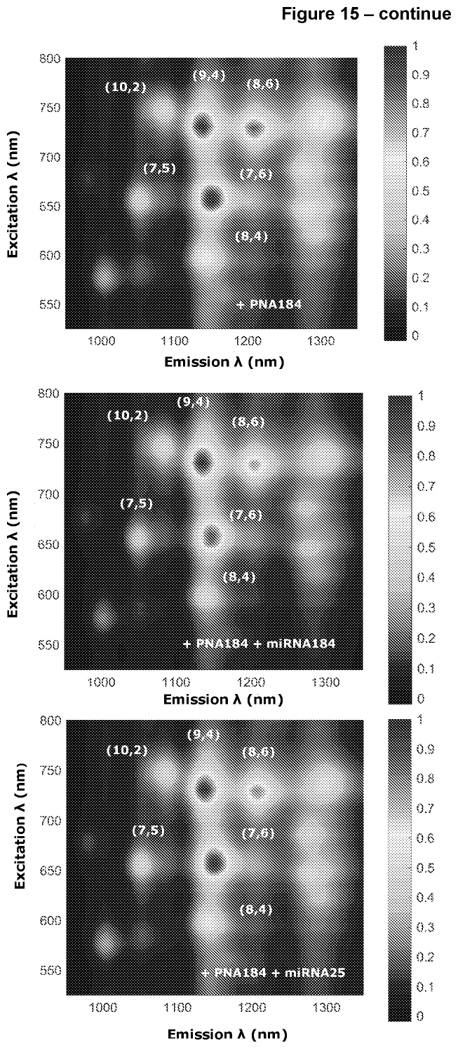

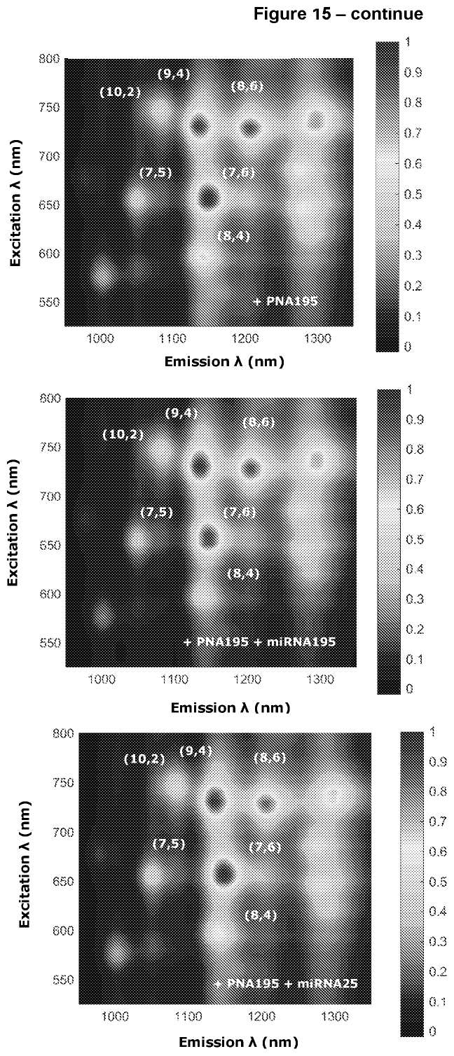

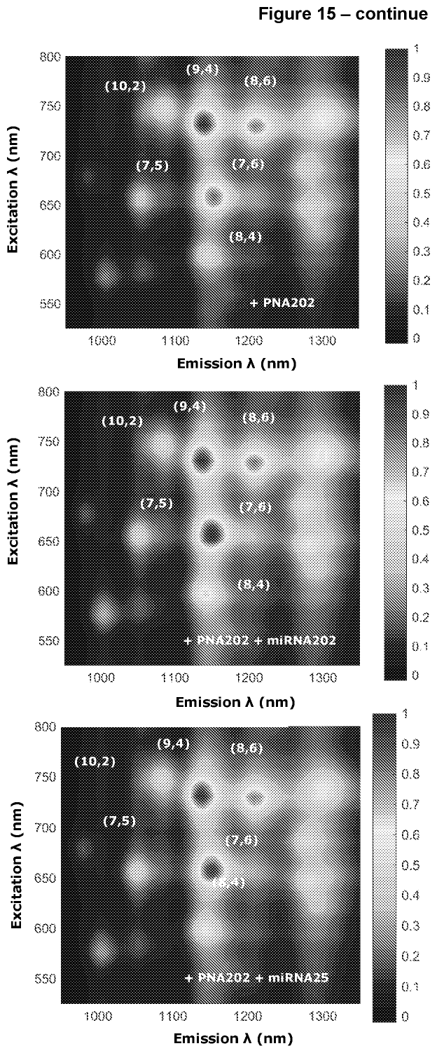

To test this hypothesis it was examined the spectral responses for constructs made using a 15 base hybrid section following the addition of higher concentrations of either complementary or non-complementary miRNA (FIG. 13, FIGS. 14, 15). As expected, no significant wavelength shift was observed for PNA10b-DNA15 bp-SWCNTs following the addition of either non-complementary miRNA sequence. Although PNA184 had strong blue-shifting responses following the addition of miR184 for both the (7,6) and (8,6) chiralities, this complex also strongly reacted with the non-complementary sequence indicating that this was not a sequence specific detection mechanism. Significant blue-shifts were also observed for all chiralities for PNA202, PNA195, and PNA155 constructs on addition of complementary miRNA.

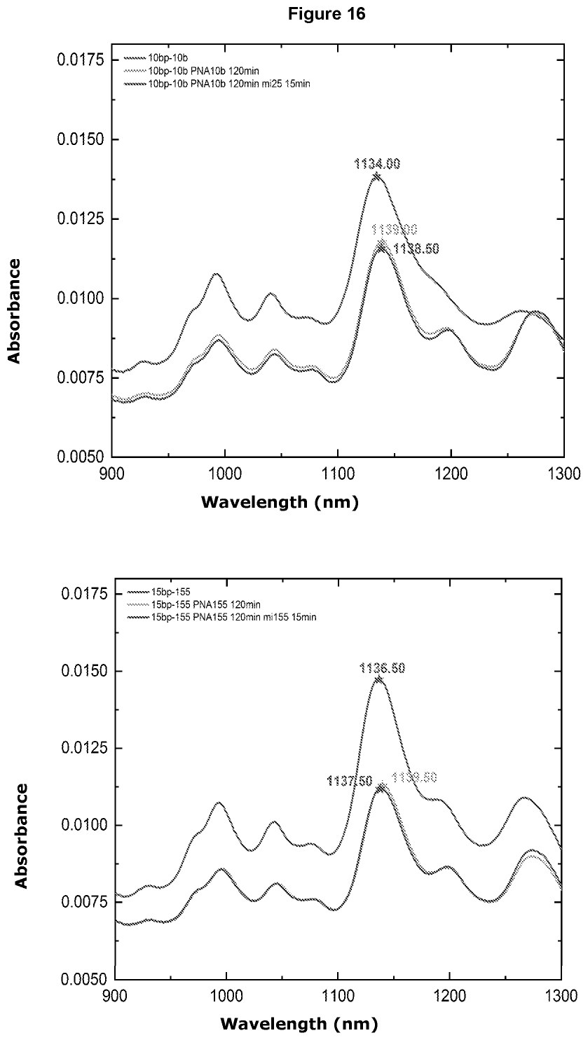

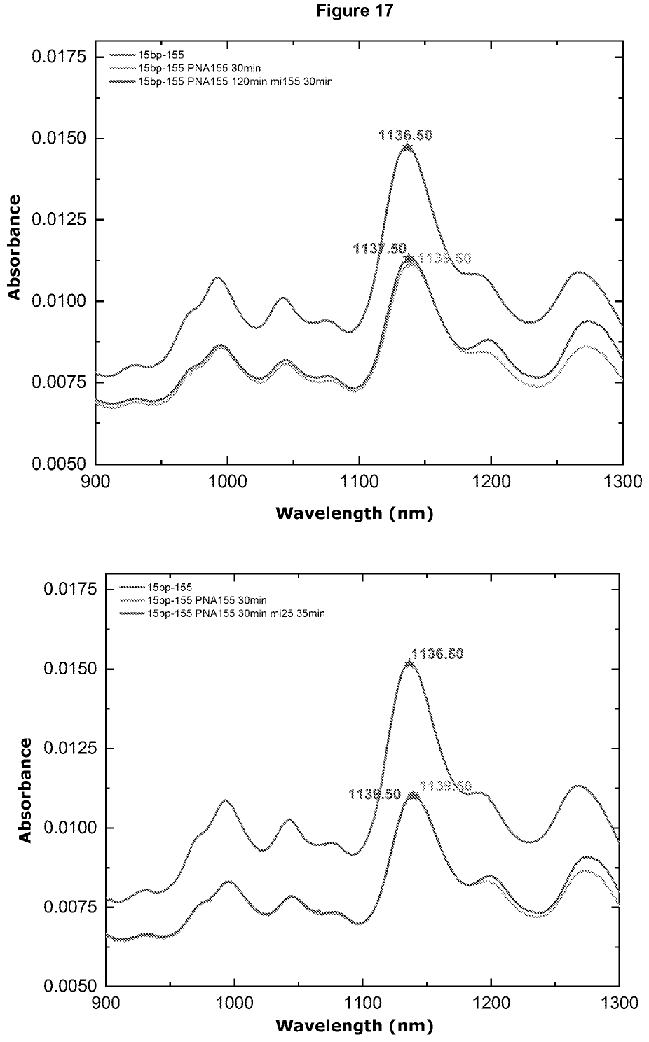

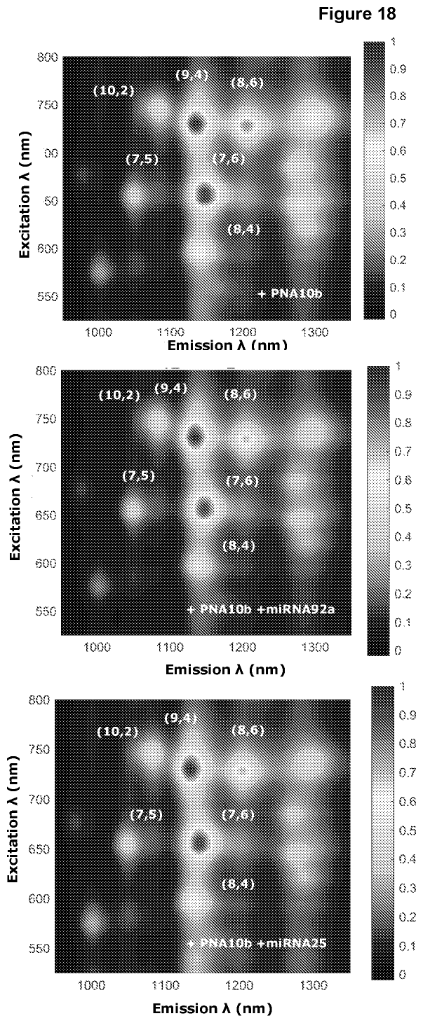

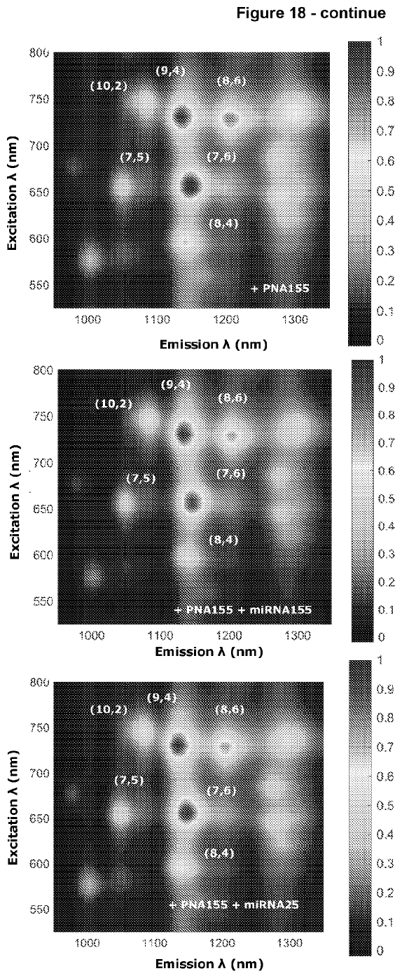

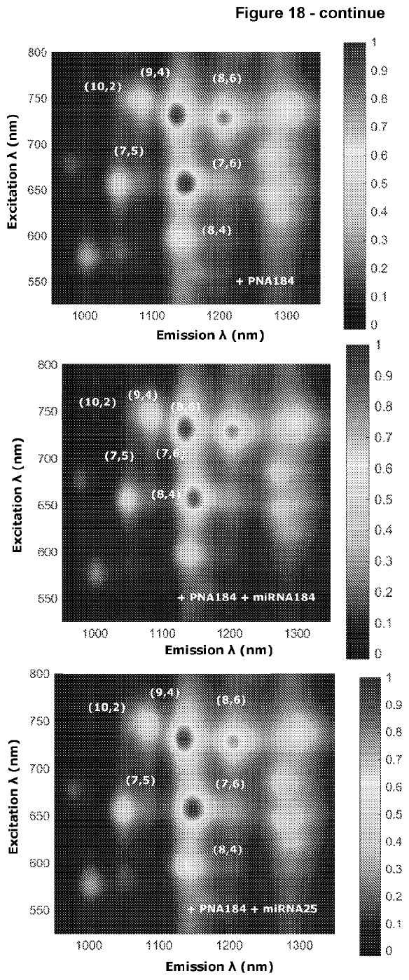

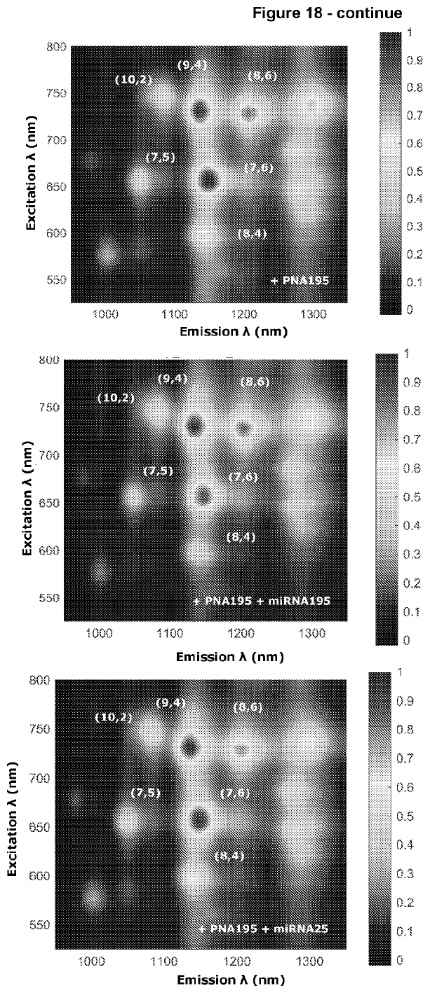

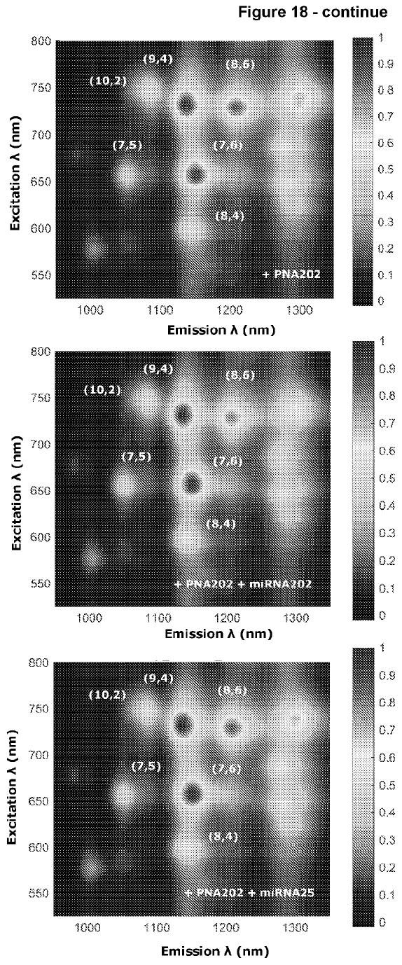

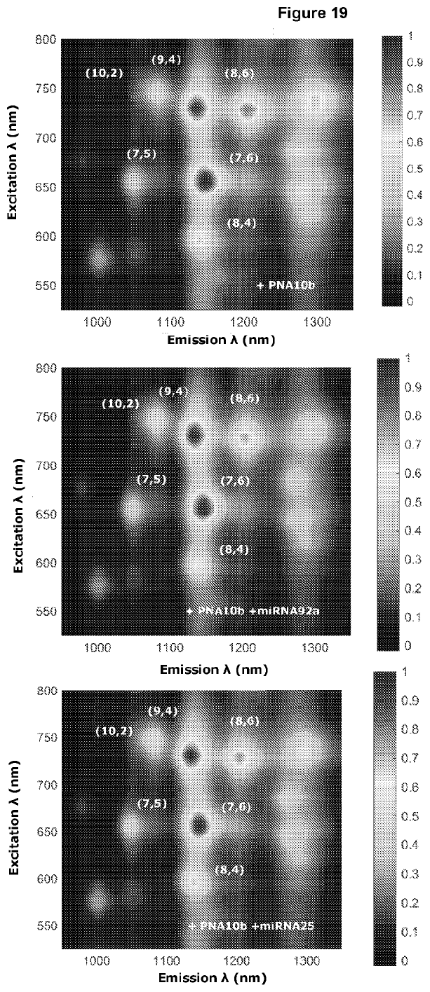

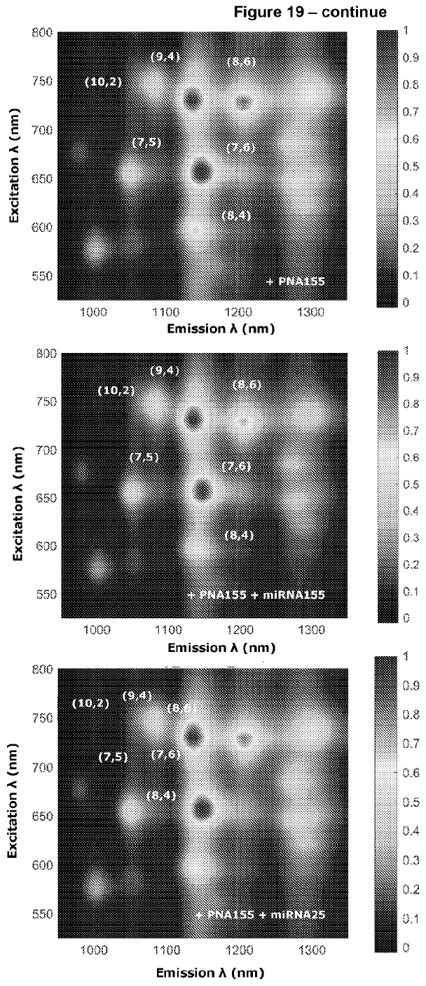

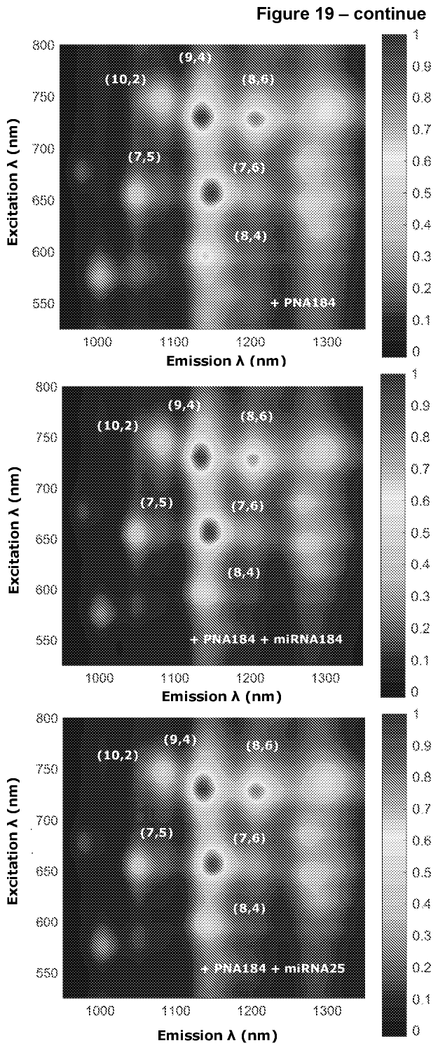

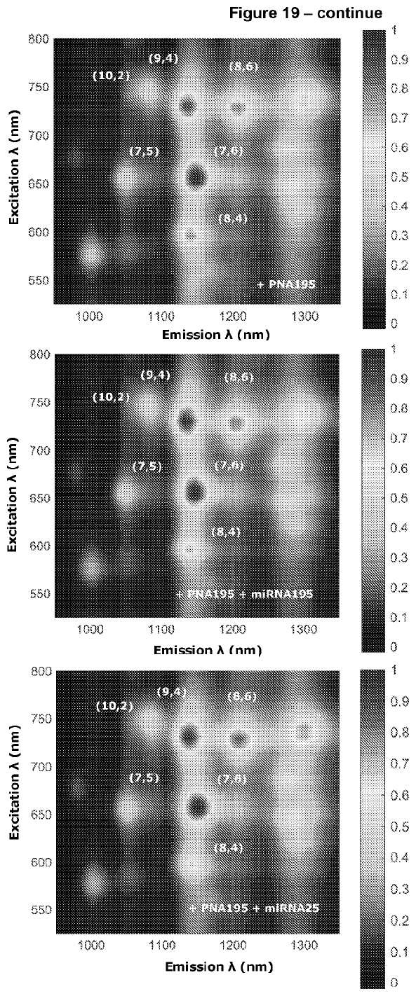

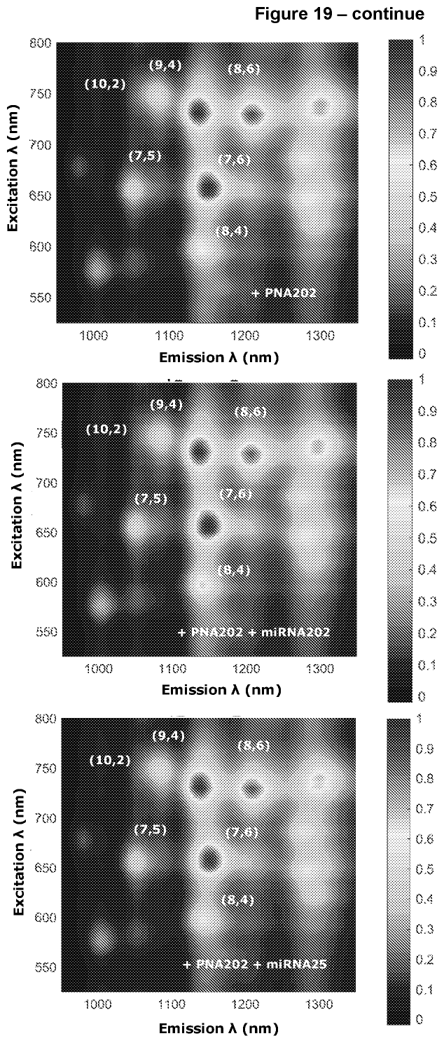

Moreover, the addition of non-complementary miRNA induced red-shifting for all PNA202 chiralities or a minor blue-shift for most PNA195 and PNA155 chiralities, indicating that the blue-shifting response to complementary miRNA was a sequence specific interaction. Also observed was a red-shift of the peak position in the absorbance spectrum (FIG. 16) of DNA15 bp-SWCNTs (1136.5 nm) on addition of PNA155 (1139.5 nm) and subsequently a blue-shift on addition of miR155 (1137.5 nm) following only 15 min of incubation (FIG. 16). No shift was observed on addition of non-complementary miRNA (FIGS. 16, 17). Additional experiments using hybrid sections with a length of 10 or 20 bases (FIGS. 18, 19, 20) also demonstrated strong blue-shifting on addition of complementary miRNA and a lack of response for non-complementary miRNA.

The novel process introduced for the assembly of PNA-DNA-SWCNTs presents a flexible platform that can be used to design sensors for many different miRNA sequences. For all constructs studied, a strong red-shift in the fluorescence of DNA-SWCNTs following the addition of PNA was observed, up to 5.5 nm depending on the DNA and PNA sequence and the nanotube chirality. On subsequent addition of miRNA, the PNA-DNA-SWCNTs underwent either blue or red-shifting. The extent and direction of shifting was strongly dependent on the PNA sequence used but also on the length of the PNA-DNA hybrid section and directionality of the hybridization. Highest shifts were observed for DNA8 bp and DNA8bp-rev at low concentrations of miRNA. It was also noted a strong dependency of the sensors on the concentration ratio of PNA:miRNA, with improved shifting responses observed as the relative concentration of miRNA was increased.

Furthermore, several of the sensors examined showed strong sequence specificity, with little to no response detected on addition of non-complementary miRNA. Based on these observations it may be possible to tune the sensor response for lower concentrations of miRNA by adjusting the relative amounts of PNA added. Using the present PNA-DNA-SWCNTs one could detect complementary miRNA additions by monitoring wavelength shifts in both absorbance and fluorescence with detection speeds of <15 min and <1 h, respectively.

### Methods

### Preparation of DNA-SWCNT Solutions

DNA-SWCNTs were prepared using a modified surfactant exchange protocol as described previously. This process involved the use of 2% SC-suspended SWCNTs, which were prepared according to the procedure outlined in Gillen et al. Briefly, 25 mg of purified HiPco-SWCNTs were added to 25 mL of SC 2% (w/v) solution. The mixture was homogenized for 20 min at 5,000 rpm (Polytron PT 1300 D, Kinematica) immediately prior to performing probe-tip sonication (¼ in. tip, Q700 Sonicator, Qsonica) for 1 h (10% amplitude) in an ice bath. The solution was centrifuged at 164,000 £ g for 4 h at 20±C (Optima XPN-80 Ultracentrifuge, Beckman) to remove any nanotube aggregates. The top 80% of the supernatant was collected and the remaining solution and pellet was discarded.

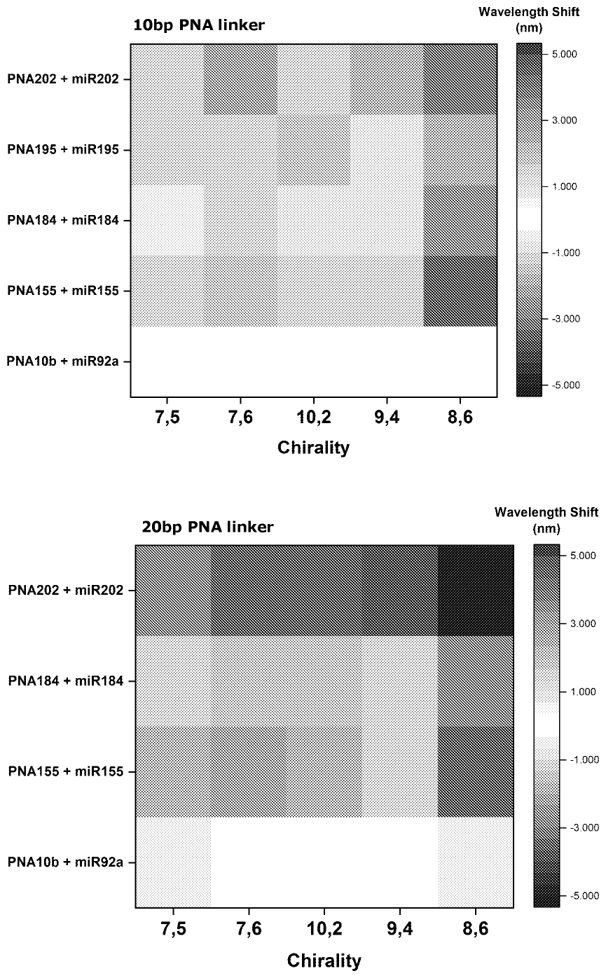

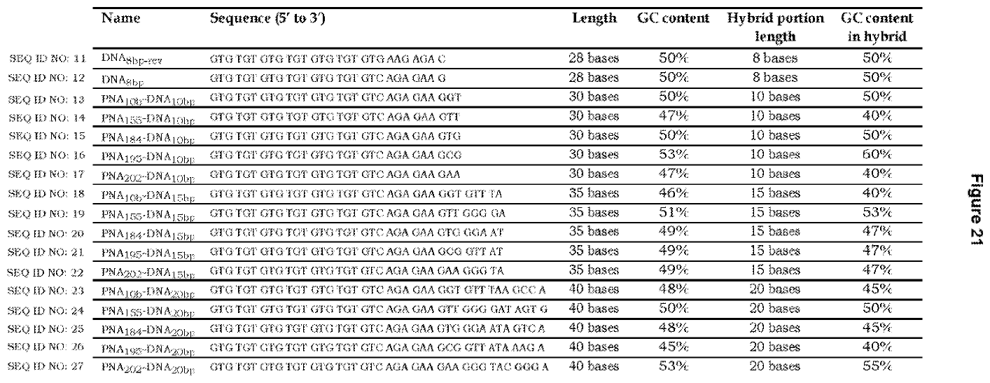

400 μL of DNA solution (33 μMin nuclease free water (QIAGEN)) was added to 400 μL of 2% SC-SWCNTs (40 mg/L) (detailed information about all the sequences used is included in FIG. 21). DNA concentrations were measured and adjusted using absorbance measurements (Nanodrop 2000, Thermo Scientific). To maintain similar starting distributions of nanotube chirality and length, the same SC-SWCNT stock was used for all suspensions. Methanol (1.2 mL) was added to the mixture to reach a final solvent percentage of 60% (v/v). The solution was vortexed briefly to mix prior incubation for at least 2 h at room temperature. Post-incubation, the DNA-SWCNTs were purified to remove all unbound DNA, displaced surfactant, and MeOH using Amicon centrifugal ultra-filtration devices (Amicon Ultra-2, Sigma Aldrich, 100 kDa membrane, Merck). Amicon devices were washed 2× as per the manufacturer's instructions prior to use with nuclease free water (4,000×g, 2 min, 20±C). SWCNT solutions were rinsed 6 times using 1 mL of nuclease free water for each wash (4,000×g, 2 min). All rinsed samples were centrifuged for 30 min at 21, 130×g and 4° C. to remove any aggregates that may have formed during the surfactant exchange protocol or washing. The top 80% of the supernatant was collected and used for subsequent experiments. All DNA-SWCNT solutions were stored at 4° C. between measurements in order mitigate aggregation of the SWCNTs.

Preparation of PNA and miRNA Solutions

PNA solutions were prepared by dissolving 50 nmoles of PNA in 500 μL of nuclease free water. Lyophilized RNA oligomers were suspended in 500 μL saline-sodium citrate (SSC) buffer (2×, stock diluted using nuclease free water). All samples were vortexed for ˜30 s to ensure the solution was homogenous. Absorbance measurements (Nanodrop 2000, Thermo Scientific) were used to adjust the concentrations of the miRNA samples to 100 μM. All miRNA solutions were subsequently divided into 25 μL aliquots and stored at −20° C. Detailed information for the miRNA and PNA sequences used in this study are presented in Table 2 and Table 3.

TABLE 2
List of miRNA sequences that were used in this study.

| | | | GC |
|---|---|---|---|
| Name | Sequence (5′ to 3′) | Length | content |
| miR92a | UAC CCU GUA GAA CCG AAU | 23 bases | 44% |
| | UUG UG (SEQ ID NO: 1) | | |
| miR155 | UUA AUG CUA AUC GUG AUA | 24 bases | 38% |
| | GGG GUU (SEQ ID NO: 2) | | |
| miR184 | UGG ACG GAG AAC UGA UAA | 22 bases | 50% |
| | GGG U (SEQ ID NO: 3) | | |
| miR195 | UAG CAG CAC AGA AAU AUU | 21 bases | 43% |
| | GGC (SEQ ID NO: 4) | | |
| miR202 | AGA GGU AUA GGG CAU GGG | 20 bases | 50% |
| | AA (SEQ ID NO: 5) | | |

TABLE 3
List of PNA sequences used in this study.

| | | | GC |
|---|---|---|---|
| Name | Sequence (N-terminus to C-terminus) | Length | content |
| PNA10b | GTC TCT TCC ACA AAT TCG GTT CTA CAG GGT | 31 bases | 45% |
| | A (SEQ ID NO: 6) | | |
| PNA155 | GTC TCT TCA ACC CCT ATC ACG ATT AGC ATT | 32 bases | 41% |
| | AA (SEQ ID NO: 7) | | |
| PNA184 | GTC TCT TCA CCC TTA TCA GTT CTC CGT CCA | 30 bases | 50% |
| | (SEQ ID NO: 8) | | |
| PNA195 | GTC TCT TCG CCA ATA TTT CTG TGC TGC TA | 29 bases | 45% |
| | (SEQ ID NO: 9) | | |
| PNA202 | GTC TCT TCT TCC CAT GCC CTA TAC CTC T | 28 bases | 50% |
| | (SEQ ID NO: 10) | | |

### Absorbance Spectroscopy

Absorbance spectra were acquired using a UV-Vis-NIR scanning spectrometer (UV-3600 Plus, Shimadzu) with samples contained in a quartz cuvette (Suprasil quartz, path length 3 mm, Hellma). All absorption spectra were collected using a 0.5 nm step size and medium scan speed. Baseline spectra were acquired with nuclease free water prior to measurement of the DNA-SWCNT samples. Concentrations were calculated using an extinction coefficient Abs632 nm=0.036 L mg⁻¹ cm⁻¹. All DNA-SWCNT solutions were diluted to 10 mg/L, corresponding to Abs632 nm=0.108, unless otherwise specified. Kinetic absorption measurements were also performed on mixtures of PNA, miRNA, and DNA-SWCNTs. A spectrum was initially collected for the DNA-SWCNT solution (67.5 μL). PNA solution (7.5 μL) was then added directly into the quartz cuvette and mixed by pipetting up and down several times. Spectra were collected immediately and then after 15, 30, 60, 90, and 120 min of incubation. miRNA solution (4.5 μL) was subsequently added and mixed by pipetting up and down. Again spectra were collected immediately and then after 15, 30, 60, 90, and 120 min of incubation.

### Fluorescence Spectroscopy Measurements

A custom-built optical set-up with an inverted Nikon Eclipse Ti-E microscope (Nikon AG Instruments) was used to acquire all fluorescence emission spectra as detailed above. Experiments were performed in 384-well plates (Clear Flat-Bottom Immuno Nonsterile 384-Well Plates, MaxiSorp, Life Technologies) which were sealed (Empore Sealing Tape Pad, 3M) prior to each fluorescence measurement to prevent evaporation. Fluorescence spectra were acquired using a 575, 660, and 745 nm laser excitation source with 10 s exposure time and 100% relative power. Spectral fitting was performed on all collected fluorescence spectra using custom Python codes. The following ratios were used for preparing the solutions prior to fluorescence measurements (nuclease free water and SSC 2× solution was added in place of PNA and miRNA for the respective controls):
45 μL of DNA-SWCNT solution (10 mg/L)+4.5 μL PNA (or nuclease free water) 100 μM+0.5 μL miRNA 100 μM (or SSC 2× solution)
45 μL of DNA-SWCNT solution (10 mg/L)+5 μL PNA (or nuclease free water) 100 μM+3 μL miRNA 100 μM (or SSC 2× solution)

Photoluminescence excitation/emission (PLE) maps were acquired between 525 nm and 800 nm using a 5 nm step and 10 s exposure time. Results were analysed using a custom Matlab code (Matlab R2017b, Mathworks).

While the invention has been disclosed with reference to certain preferred embodiments, numerous modifications, alterations, and changes to the described embodiments, and equivalents thereof, are possible without departing from the sphere and scope of the invention. Accordingly, it is intended that the invention not be limited to the described embodiments, and be given the broadest reasonable interpretation in accordance with the language of the appended claims.

1. A molecular tool comprising:
(i) a substrate comprising or consisting of an optical material;
(ii) a first polymeric molecule comprising a nucleic acid or a nucleic acid analogue, wherein the first polymeric molecule is coupled with the substrate; and
(iii) a second polymeric molecule coupled with the first polymeric molecule by means of nucleobase complementarity, wherein the second polymeric molecule comprises a nucleic acid analogue.

2. The molecular tool according to claim 1, wherein the optical material comprises Carbon Nanotubes (CNTs), Single Wall Carbon Nanotubes (SWCNTs), graphene, quantum dots, graphene quantum dots, plasmonically enhanced nanoparticles, or fluorophores.

3. The molecular tool according to claim 1, wherein the optical material is Single Wall Carbon Nanotubes (SWCNTs).

4. The molecular tool according to claim 1, wherein the optical material is a stable dispersion of isolated Single Wall Carbon Nanotubes (SWCNTs).

5. The molecular tool according to claim 1, wherein the first polymeric molecule comprises a nucleic acids.

6. The molecular tool according to claim 1, wherein the nucleic acid is a deoxyribonucleic acids (DNA) or a ribonucleic acids (RNA).

7. The molecular tool according to claim 1, wherein the nucleic acid analogue comprises a glycerol nucleic acid (GNA), threose nucleic acid (TNA), locked nucleic acid (LNA), peptide nucleic acids (PNAs), cyclohexenyl nucleic acid (CeNA), or hexose nucleic acid (HNA).

8. The molecular tool according to claim 1, wherein the second polymeric molecule is coupled with the first polymeric molecule by partial overlap.

9. (canceled)

10. A label-free method for detecting a target nucleic acid in a sample, the method comprising the steps of:
(i) contacting a sample with the molecular tool according to claim 1 under conditions allowing the coupling by means of nucleobase complementarity between the second polymeric molecule of the molecular tool and the target nucleic acid in the sample;
(ii) providing an electromagnetic radiation to the sample comprising the molecular tool under conditions allowing the interaction of the electromagnetic radiation with the optical material of the molecular tool; and
(ii) collecting and analyzing an optical signal derived from the optical material of the molecular tool, wherein the optical signal is indicative of the coupling between the second polymeric molecule and the target nucleic acid in the sample.

11. The method according to claim 10, wherein the target nucleic acid comprises cell-free tumor DNA, circulating mRNA, or circulating microRNA (miRNA).

12. The method according to claim 10, wherein the sample is a biological sample comprising blood, plasma, saliva, urine, biopsy, an organ, a tissue or a cell sample.

13. The method according to claim 10, wherein the sample is a native, unprocessed biological sample.

14. The method according to claim 10, wherein the electromagnetic radiation comprises visible light, UV light or IR light.

15. The method according to claim 10, wherein the optical signal comprises fluorescence or fluorescence wavelength shift.

16. The method according to claim 10, wherein the step of collecting and analyzing an optical signal comprises performing a photoluminescence excitation/emission spectroscopy analysis in the near IR spectrum.

17. A kit comprising at least one molecular tool according to claim 1, at least one container, and instructions for use.

18. A system for detecting a target nucleic acid sequence in a sample, the system comprising the molecular tool according to claim 1, a source of electromagnetic radiation, and an electromagnetic radiation detector for collecting and analyzing an optical signal derived from the optical material of the molecular tool.

19. The system according to claim 18, wherein the target nucleic acid comprises cell-free tumor DNA, circulating mRNA, or circulating microRNA (miRNA).

20. The system according to claim 18, wherein the sample is a biological sample comprising blood, plasma, saliva, urine, a biopsy, an organ, a tissue or a cell sample.

21. The system according to claim 18, wherein the sample is a native, unprocessed biological sample.

22. The system according to claim 18, wherein the electromagnetic radiation comprises visible light, UV light or IR light.

23. The system according to claim 18, wherein the optical signal comprises fluorescence or fluorescence wavelength shift.

24. The system according to claim 18, wherein the collecting and analyzing an optical signal comprises performing a photoluminescence excitation/emission spectroscopy analysis in the near IR spectrum.

25. The molecular tool according to claim 6, wherein the nucleic acid is a deoxyribonucleic acid (DNA).

26. The molecular tool according to claim 7, wherein the nucleic acid analogue is a peptide nucleic acid (PNA).

27. The method according to claim 11, wherein the target nucleic acid in the sample is a miRNA.

28. The system according to claim 19, wherein the target nucleic acid in the sample is a miRNA.


SEQUENCE LISTING (27 sequences)

SEQ ID NO: 1 - RNA, 23 nt, Artificial Sequence
  synthetic sequence
uacccuguagaaccgaauuugug

SEQ ID NO: 2 - RNA, 24 nt, Artificial Sequence
  synthetic sequence
uuaaugcuaaucgugauagggguu

SEQ ID NO: 3 - RNA, 22 nt, Artificial Sequence
  synthetic sequence
uggacggagaacugauaagggu

SEQ ID NO: 4 - RNA, 21 nt, Artificial Sequence
  synthetic sequence
uagcagcacagaaauauuggc

SEQ ID NO: 5 - RNA, 20 nt, Artificial Sequence
  synthetic sequence
agagguauagggcaugggaa

SEQ ID NO: 6 - DNA, 31 bp, Artificial Sequence
  synthetic sequence
gtctcttccacaaattcggttctacagggta

SEQ ID NO: 7 - DNA, 32 bp, Artificial Sequence
  synthetic sequence
gtctcttcaacccctatcacgattagcattaa

SEQ ID NO: 8 - DNA, 30 bp, Artificial Sequence
  synthetic sequence
gtctcttcacccttatcagttctccgtcca

SEQ ID NO: 9 - DNA, 29 bp, Artificial Sequence
  synthetic sequence
gtctcttcgccaatatttctgtgctgcta

SEQ ID NO: 10 - DNA, 28 bp, Artificial Sequence
  synthetic sequence
gtctcttcttcccatgccctatacctct

SEQ ID NO: 11 - DNA, 28 bp, Artificial Sequence
  synthetic sequence
gtgtgtgtgtgtgtgtgtgtgaagagac

SEQ ID NO: 12 - DNA, 28 bp, Artificial Sequence
  synthetic sequence
gtgtgtgtgtgtgtgtgtgtcagagaag

SEQ ID NO: 13 - DNA, 30 bp, Artificial Sequence
  synthetic sequence
gtgtgtgtgtgtgtgtgtgtcagagaaggt

SEQ ID NO: 14 - DNA, 30 bp, Artificial Sequence
  synthetic sequence
gtgtgtgtgtgtgtgtgtgtcagagaagtt

SEQ ID NO: 15 - DNA, 30 bp, Artificial Sequence
  synthetic sequence
gtgtgtgtgtgtgtgtgtgtcagagaagtg

SEQ ID NO: 16 - DNA, 30 bp, Artificial Sequence
  synthetic sequence
gtgtgtgtgtgtgtgtgtgtcagagaagcg

SEQ ID NO: 17 - DNA, 30 bp, Artificial Sequence
  synthetic sequence
gtgtgtgtgtgtgtgtgtgtcagagaagaa

SEQ ID NO: 18 - DNA, 35 bp, Artificial Sequence
  synthetic sequence
gtgtgtgtgtgtgtgtgtgtcagagaaggtgttta

SEQ ID NO: 19 - DNA, 35 bp, Artificial Sequence
  synthetic sequence
gtgtgtgtgtgtgtgtgtgtcagagaagttgggga

SEQ ID NO: 20 - DNA, 35 bp, Artificial Sequence
  synthetic sequence
gtgtgtgtgtgtgtgtgtgtcagagaagtgggaat

SEQ ID NO: 21 - DNA, 35 bp, Artificial Sequence
  synthetic sequence
gtgtgtgtgtgtgtgtgtgtcagagaagcggttat

SEQ ID NO: 22 - DNA, 35 bp, Artificial Sequence
  synthetic sequence
gtgtgtgtgtgtgtgtgtgtcagagaagaagggta

SEQ ID NO: 23 - DNA, 40 bp, Artificial Sequence
  synthetic sequence
gtgtgtgtgtgtgtgtgtgtcagagaaggtgtttaagcca

SEQ ID NO: 24 - DNA, 40 bp, Artificial Sequence
  synthetic sequence
gtgtgtgtgtgtgtgtgtgtcagagaagttggggatagtg

SEQ ID NO: 25 - DNA, 40 bp, Artificial Sequence
  synthetic sequence
gtgtgtgtgtgtgtgtgtgtcagagaagtgggaatagtca

SEQ ID NO: 26 - DNA, 40 bp, Artificial Sequence
  synthetic sequence
gtgtgtgtgtgtgtgtgtgtcagagaagcggttataaaga

SEQ ID NO: 27 - DNA, 40 bp, Artificial Sequence
  synthetic sequence
gtgtgtgtgtgtgtgtgtgtcagagaagaagggtacggga